SEQUENCE LISTING (2 sequences)

SEQ ID NO: 1 - DNA, 21 bp, synthetic construct
  source 1..21: mol_type = other DNA
caaatagtttccctaatcatc

SEQ ID NO: 2 - DNA, 21 bp, synthetic construct
  source 1..21: mol_type = other DNA
ctacgggtgcatcagataatt

(19) United States
(12) Patent Application Publication
(10) Pub. No.: US 2026/0137671 A1
(43) Pub. Date: May 21, 2026

(54) PDK4 as a Cell-Aging Intervention Target and Its Use in Chemotherapy and Anticancer

(71) Applicant: Shanghai Institute of Nutrition and Health, Chinese Academy of Sciences, Shanghai (CN)

(72) Inventor: Yu Sun, Shanghai (CN)

(21) Appl. No.: 18/697,539

(22) PCT Filed: Dec. 7, 2023
(86) PCT No.: PCT/CN2023/137044

§ 371 Date: Apr. 1, 2024

(30) Foreign Application Priority Data
Dec. 23, 2022 (CN) 202211705625.9

(51) Int. Cl. A61K 31/454; A61K 31/136; A61K 31/282; A61K 31/337; A61K 31/475; A61K 31/513; A61K 31/704; A61K 31/7068; A61K 33/243; A61K 38/14; A61P 35/00; C12Q 1/6886; G01N 33/575

(52) U.S. Cl. CPC A61K 31/454; A61K 31/136; A61K 31/282; A61K 31/337; A61K 31/475; A61K 31/513; A61K 31/704; A61K 31/7068; A61K 33/243; A61K 38/14; A61P 35/00; C12Q 1/6886; G01N 33/5758; C12Q 2600/106; C12Q 2600/158; G01N 2333/912

(57) ABSTRACT

The present disclosure provides Pyruvate Dehydrogenase Kinase 4 (PDK4) as a target for intervention of cellular senescence and a use in chemotherapy against cancer. The present disclosure reveals PDK4 as a novel target for intervention of cellular senescence and intervention in drug resistance of tumors. By targeting PDK4, drugs can be developed to inhibit drug resistance of tumors, suppress or eliminate the SASP. Additionally, drugs targeting PDK4 can be used in combination with chemotherapeutic drugs for diagnosis or prognosis of related diseases or symptoms based on this target and mechanism.

The present disclosure claims the benefit of priority to Patent Application CN202211705625.9, named “PDK4 as a target for intervention of cellular senescence and a use in chemotherapy against cancer” and filed Dec. 23, 2022, with its entire content incorporated herein by reference.

### TECHNICAL FIELD

The present disclosure belongs to the field of biomedicine, and more specifically, the present disclosure refers to PDK4 as a target for cellular senescence intervention and a use in chemotherapy against cancer.

### TECHNICAL BACKGROUND

Senescence is a complex biological process characterized by tissue homeostasis imbalance and functional degeneration, accompanied by a significant increase in the incidence of senescence-associated diseases. Senescence is closely associated with mitochondrial dysfunction, metabolic dysregulation and chronic inflammation. Additionally, the number reduction and dysfunction of stem cells, changes that accompany the aging process, can lead to impaired tissue regeneration and homeostasis, contributing to tissue and organismal aging at multiple levels.

Numerous studies have indicated that cellular senescence is the primary driving force and fundamental block of organismal aging. Cellular senescence is a process, wherein cells undergo a permanent cycle-arrest. Cellular senescence is often associated with various pathological characteristics, including local inflammation. Cellular senescence occurs in damaged cells and prevents their continued proliferation within the organism. Under the influence of various external stimuli and internal stresses, cell damage can lead to pronounced signs of cellular senescence. Moreover, when damage accumulates and reaches a certain threshold, tissues exhibit visible degenerative changes and aging-associated phenotypes at the physiological level.

The concept of senescence-associated secretory phenotype (SASP) was first proposed by Coppe, and colleagues in 2008. They found that senescent cells can promote the malignancy of adjacent precancerous cells by secreting extracellular matrix proteins, inflammation-related factors and cancer cell growth factors, which are collectively referred to as SASP factors. In the intracellular environment, senescent cells accumulate in various organs, accompanied by a complex set of SASP factors. It is a phenotype characterized by significantly enhanced intracellular expression and extracellular release of various types of cytokines, making it a crucial biochemical and cellular property of senescent cells. SASP factors cover pro-inflammatory cytokines (such as IL-1α, IL-1β, IL-6, and IL-8), growth factors (such as HGF, TGF-β, and GM-CSF), various chemokines (such as CXCL1/3 and CXCL10), matrix remodeling enzymes (such as MMP1 and MMP3), and so on. The diverse biological activities induced by the components of SASP suggest its involvement in intricate multi-ligand and multi-receptor signaling mechanisms that regulate the local tissue's microenvironment, with a potential to contribute to or exacerbate age diseases, including malignancies.

A typical feature of cancer cells is their ability to reprogram energy metabolism to promote proliferation and survival per se, with enhanced mitochondrial function playing a crucial role in tumor progression. One major property of senescent cells is their maintenance of active metabolism and synthesis of a large amount of SASP factors, which can locally or systemically impact the microenvironment of host cells. Previous researches on the metabolism of senescent cells have shown an increase in glucose consumption and lactate production during aging. Glucose is the primary energy source for senescent cells, but the glucose metabolic characteristics of stroma and non-cancerous tissues, as well as the impact of their metabolic spectrum on the homeostasis of surrounding tissues still remain largely unclear. Therefore, unraveling the regulatory mechanisms of cellular senescence and its specific phenotypes from a metabolic perspective, and subsequently revealing key target molecules and signaling pathways, represents emerging directions in the field of aging biology and geriatric medicine. In-depth exploration is urgently needed to provide important scientific foundations and potential interventions for clinical medicine.

### SUMMARY OF THE INVENTION

The aim of the present disclosure is to propose pyruvate dehydrogenase kinase 4 (PDK4) as a target for intervention of cellular senescence and a use in chemotherapy against cancer. The present disclosure reveals PDK4 as a novel target for intervention of cellular senescence and intervention in cancer resistance. By targeting PDK4, agents can be developed to inhibit drug resistance of tumors, suppress or eliminate the SASPSASP. Additionally, agents targeting PDK4 can be used in combination with chemotherapeutic drugs for diagnosis or prognosis of related diseases or symptoms based on the relevant target and mechanism.

In the first aspect, the present disclosure provides a use of a PDK4 down-regulator, for:
preparing a composition for inhibiting drug resistance of tumors;
preparing a composition for inhibiting tumors or suppressing drug resistance of tumors combined with chemotherapeutic drugs; or preparing a composition for inhibiting or eliminating the SASPSASP.

In the second aspect, the present disclosure provides a use of PDK4, for:
serving as a target (or biomarker) to inhibit drug resistance of tumors or screen drugs for suppressing drug resistance of tumors;
serving as a target (or biomarker) to inhibit or eliminate SASP the SASP or screen drugs for inhibiting or eliminating SASP the SASP;
acting as a biomarker for diagnosing or predicting drug resistance of tumors; or preparing a diagnostic reagent for diagnosis or prognosis of drug resistance of tumors.

In the third aspect, the present disclosure provides a composition, a pharmaceutical composition or a drug kit for inhibiting tumors, comprising: a down-regulator of PDK4 and a chemotherapeutic drug.

In one or more embodiments, the tumor is a tumor with drug resistance; preferably comprising a tumor with drug resistance after exposure to genotoxic drugs or exhibiting SASP the SASP in the tumor microenvironment; more preferably, comprising: prostate cancer, breast cancer, lung cancer, colorectal cancer, gastric cancer, liver cancer, pancreatic cancer, bladder cancer, skin cancer, kidney cancer.

In one or more embodiments, the SASP the SASP is a SASP caused by DNA damage; preferably, the DNA damage is a DNA damage caused by a chemotherapeutic drug; more preferably, the chemotherapeutic drug comprises a genotoxic drug.

In one or more embodiments, the SASP SASP is a hypermetabolic phenotype, preferably the hypermetabolic phenotype is a phenotype with enhanced glycolysis, and/or increased TCA activities, and/or enhanced ATP-boosting oxidative phosphorylation; more preferably, the hypermetabolic phenotype is a phenotype with an increased citrate/pyruvate ratio, elevated levels of metabolites (preferably, comprising dihydroxyacetone, 3-phosphoglyceraldehyde, pyruvate, alanine, lactate, 3-phosphoglyceric acid, citrate, α-ketoglutarate, glutamate, succinate, succinate, fumarate or malate), increased expression or activity of enzymes (preferably, comprising GLUT1, HK2, LDHA, IDH2, IDH3, OGDH, CS) in metabolic processes, perturbations in mitochondrial ultrastructure, significantly increased extracellular acetone and lactate production and extracellular acidification rate (ECAR), increased oxygen consumption rate (OCR), elevated ATP production, basal respiration and maximal respiration.

In one or more embodiments, the down-regulatordown-regulator of PDK4 comprises a substance down-regulating PDK4 activities or a substance down-regulating expression levels of PDK4, increasing the stability of PDK4 or reducing the effective time of PDK4;
preferably, the down-regulator comprises: a chemical molecular antagonist or inhibitor targeting PDK4, a reagent knocking out or silencing PDK4, or a proteasome;
more preferably, the down-regulator of PDK4 is PDK4-IN; or the reagent knocking out or silencing PDK4 comprises: an interfering molecule that specifically interfering the expression of the encoding sequence of PDK4, a CRISPR gene-editing agent targeting PDK4, a homologous recombinant agent or an agent for site-directed mutagenesis, wherein the agent for site-directed mutagenesis brings a loss-of-function mutation to the PDK4.

In one or more embodiments, the chemotherapeutic drug is a chemotherapeutic drug induces drug resistance of tumor after administration; preferably comprising a genotoxic drug; more preferably comprising: mitoxantrone, doxorubicin, bleomycin, vinblastine, paclitaxel, docetaxel, satraplatin, cisplatin, carboplatin, daunorubicin, nogamycin, aclarubicin, epirubicin, doxorubicin, cytarabine, capecitabine, gemcitabine or 5-fluorouracil.

In one or more embodiments, the weight ratio of PDK4 down-regulator to chemotherapeutic drug is 1:1˜1:200, preferably 1:2˜1:100, more preferably 1:5˜1:50.

In one or more embodiments, the final concentration of PDK4 down-regulator is 0.1-100 M, preferably 0.5˜50 M, more preferably 1˜10 M; or the final concentration of the chemotherapeutic drug is 0.01˜100 M, preferably 0.05˜80 M, more preferably 0.1˜50 M.

In one or more embodiments, the chemotherapeutic drug is mitoxantrone, wherein the final concentration of the chemotherapeutic drug is 0.01˜10 M, 0.05˜5 M, 0.1˜1 M; for example, 0.1 M, 0.2 M, 0.3 M, 0.4 M, 0.5 M, 0.6 M, 0.7 M, 0.8 M, 0.9 M, 1.0 M; the final concentration of the PDK4 down-regulator is 0.1˜100 M, 0.5˜50 M, 1˜10 M; for example, 1 M, 2 M, 3 M, 4 M, 5 M, 6 M, 7 M, 8 M, 9 M, 10 M.

In one or more embodiments, the dosage is a dosage administered to mice. It is known for those skilled in the art that the dosage of mice can be converted into dosages suitable for other animals or humans.

In one or more embodiments, the dosage of PDK4 down-regulator (for example, PDK4-IN) is 1˜100 mg/kg, preferably 2˜50 mg/kg, more preferably 5˜20 mg/kg; and the dosage of the chemotherapeutic drug is 0.01˜10 mg/kg, preferably 0.05˜5 mg/kg, more preferably 0.1˜1 mg/kg.

In one or more specific embodiments, the dosage of the chemotherapeutic drug mitoxantrone (MIT) is 0.01˜10 mg/kg, 0.05˜5 mg/kg, 0.1˜1 mg/kg; for example, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg.

In one or more specific embodiments, the dosage of the chemotherapeutic drug doxorubicin (DOX) is 0.01˜10 mg/kg, 0.05˜5 mg/kg, 0.05˜3 mg/kg, 0.1˜2 mg/kg, 0.1˜1 mg/kg; for example, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg.

In one or more specific embodiments, the dosage of the chemotherapeutic drug paclitaxel (DTX) is 0.01˜10 mg/kg, 0.05˜5 mg/kg, 0.05˜3 mg/kg, 0.1˜2 mg/kg, 0.1˜1 mg/kg; for example, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg.

In one or more specific embodiments, the dosage of the chemotherapeutic drug docetaxe (PTX) is 0.01˜10 mg/kg, 0.05˜5 mg/kg, 0.05˜3 mg/kg, 0.1˜2 mg/kg, 0.1˜1 mg/kg; for example, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg.

In the fourth aspect, the present disclosure provides a method for preparing a pharmaceutical combination or drug kit for inhibiting tumors, comprising: mixing the PDK4 down-regulator and the chemotherapeutic drug; or placing the PDK4 down-regulator and the chemotherapeutic drug in the same drug kit.

In the fifth aspect, the present disclosure provides a method for screening a potential substance for inhibiting the SASP, wherein the method comprises:
(1) providing a system of tumor microenvironment, wherein the system comprises tumor cells and stromal cells;
(2) treating the system of (1) with a chemotherapeutic drug, inducing a SASP in the tumor microenvironment;
(3) adding the candidate substance to the system in (2) and observing its effect on the tumor microenvironment system;
if it shows specifically-targeted clearance of senescent cells in a tumor microenvironment and/or promotes the growth of stromal cells (non-senescent cells) (increases the proliferation rate of stromal cells), then it is a potential substance for enhancing the anti-tumor effect of chemotherapeutic drugs;
preferably,
in step (2), it also comprises: administering a PDK4 down-regulator before, during or after inducing the SASP (for example, hypermetabolic phenotype) in the tumor microenvironment;
and/or, in step (3), it also comprises: if the candidate substance statistically promotes a down-regulator of PDK4 or an encoding gene thereof to clear senescent cells in the tumor microenvironment and/or promotes the growth of stromal cells, then the candidate substance is a potential substance that can be used in combination with the down-regulator of PDK4 or the encoding gene thereof to inhibit tumors.

In the sixth aspect, the present disclosure provides a method for screening a potential substance for inhibiting drug resistance of tumors or inhibiting the SASP, wherein the method comprises:
(1) treating an expression system by a candidate substance, wherein the system expressing PDK4; and
(2) detecting the system to observe the expression or activity of PDK4; if the expression or activity of PDK4 is down-regulated (obviously down-regulated, such as down-regulated by at least 10%, at least 20%, at least 50%, at least 80%, and so on; or with a silenced expression or without activities) statistically by the candidate substance, then the candidate substance is a potential substance for inhibiting drug resistance of tumors or inhibiting the SASP;
preferably, the step (1) comprises: in the testing group, adding the candidate substance into the expression system; and/or
the step (2) comprises: detecting the system to observe the expression or activity of PDK4 and compared to the control group, wherein the control group is an expression system without adding the candidate substances; if the expression or activity of PDK4 is down-regulated statistically by the candidate substance, then the candidate substance is the potential substance for inhibiting drug resistance of tumor or inhibiting the SASP.

In one or more embodiments, the candidate substances include (but are not limited to): regulatory molecules designed for PDK4 or its upstream or downstream proteins or genes, small molecular compounds, and so on.

In one or more embodiments, the system is selected from: cellular systems (such as cells expressing PDK4 or cell cultures), subcellular systems, solution systems, tissue systems, organ systems or animal systems.

In one or more embodiments, the method also comprises: further cell experiments and/or animal experiments on the obtained potential substances, so as to further select and determine substances really useful for inhibiting drug resistance of tumor or inhibiting/eliminating the SASP.

In the seventh aspect, the present disclosure provides a use of a reagent specifically recognizing or amplifying PDK4, or a reagent specifically recognizing or detecting lactate, for preparing diagnostic reagents or kits for diagnosing or predicting drug resistance of tumor.

In one or more embodiments, the diagnostic reagents comprise: a binding molecule specifically binding to PDK4 protein, a primer specifically amplifying the PDK4 gene, a probe specifically recognizing the PDK4 gene, a chip specifically recognizing the PDK4 gene, a binding molecule specifically binding to lactate, a probe specifically recognizing lactate, or an enzyme specifically monitoring (detecting) lactate levels.

In the eighth aspect, the present disclosure provides a use of a PDK4 down-regulator for preparing a drug or a formulation, wherein the drug or the formulation is used for: downregulating the SASP, downregulating hypermetabolic phenotypes, decreasing levels of glycolysis, reducing TCA activities, inhibiting ATP-boosting oxidative phosphorylation, decreasing levels of metabolites, inhibiting enzyme expression or activity in metabolic processes, inhibiting extracellular release of metabolites, inhibiting ATP production, reducing respiratory rates, eliminating senescence, extending the lifespan of the subject, reducing the burden of age-related diseases in the subject, preventing, alleviating and treating diseases benefiting from the reduction or elimination of non-proliferating cells, reducing resistance to cancer therapy, or enhancing the effects of agents inducing cellular senescence;
preferably, the respiratory rates comprise: basal respiratory rate, maximal respiratory rate;
and/or, the metabolites comprise: dihydroxyacetone, 3-phosphoglyceraldehyde, pyruvate, alanine, lactate, 3-phosphoglyceric acid, citrate, α-ketoglutarate, glutamate, succinate, succinate, fumarate or malate;
and/or, the enzymes in the metabolic process comprise: GLUT1, HK2, LDHA, IDH2, IDH3, OGDH or CS.

Other aspects of the present disclosure will be apparent to those skilled in the art based on the disclosure herein.

### DESCRIPTION OF FIGURES

FIG. 1. Analysis of gene expression at transcript level of primary human stromal cell line PSC27 by gene chip (microarray). On the 7th day post-treatment, cell lysates were collected for analysis. CTRL, control. RAD, radiation. BLEO, bleomycin. HP, H₂O₂. Red asterisk indicates typical SASP soluble factors. Arrow indicates PDK4.

FIG. 2. Quantitative RT-PCR detection was used to determine PDK4 expression after separate treatment of PSC27. After establishing stable cell subpopulations or completing in vitro treatments for 7 days, cell lysates were collected for analysis. Signals are normalized to CTRL. RS, replicative senescence. p16, lentiviral transduction of human tumor suppressor gene p16^{INK4a} RAS, lentiviral transduction of human oncogene HRAS^{G12V}.

FIG. 3. Immunoblot measurement of PDK4 expression in stromal cells. GAPDH, protein loading control.

FIG. 4. Comparative expressions of PDK4 in PSC27 or prostate epithelial cells after drug treatment. On the 7th day post-treatment, cell lysates were collected for analysis. Signals are normalized to CTRL. BPH1, M12, PC3, DU145, LNCAP and VCAP, epithelial cancer cell lines of human prostate origins.

FIG. 5. Comparative RT-PCR expressions of PDK4 in human stromal cells after treatment for 7 days. WI38, HFL1, HBF1203 and BJ, human stromal cell lines of different origins.

FIG. 6. A time-course RT-PCR assessment of the expression of PDK and a set of typical SASP factors (MMP1, WNT16B, SFRP2, SPINK1, MMP3, CXCL8, EREG, ANGPTL4, and AREG) after in vitro drug treatment of PSC27 cells. Numeric numbers indicate the individual days after treatment (indexed).

FIG. 7. Immunoblot measurement of PDK4 expression at the protein level at the individual time points. β-actin, protein loading control.

FIG. 8. Comparative appraisal of human PDK family (1-4) expression at transcript level in PSC27 cells after treatment. Signals are normalized to untreated sample per gene. CXCL8, experimental control as a hallmark SASP factor. β-actin, protein loading control. Arrow indicates PDK4.

FIG. 9. Representative images showing PDK4 expression in samples of patients with prostate cancer. Left, immunohistochemical (IHC) staining. Right, hematoxylin-eosin (HE) staining. Left, untreated; Right, chemo-treated. The rectangular region selected in the upper image of each staining is magnified in the lower image. Scale bar, 100 μm.

FIG. 10. Pathological evaluation of stromal PDK4 expression in PCa samples (untreated, 42 cases; treated, 48 cases). Patients were classified into four categories based on the immunohistochemical (IHC) staining intensity of PDK4 in the stroma. 1, negative; 2, weak; 3, moderate; 4, strong expression. Left, statistical comparison. Right, representative images of PDK4 signals in each category. EL, expression level. Scale bar, 100 μm.

FIG. 11. Boxplot summary of PDK4 transcript expression by qRT-PCR analysis upon laser capture microdissection (LCM) of cells from tumor and stroma, respectively. Signals normalized to the lowest value in untreated epithelium group, with comparison performed between untreated (42 cases) and treated (48 cases) samples per cell lineage. For epithelial or stromal-derived cells, samples from 10 untreated and treated patients in each group were randomly selected for further analysis and parallel comparison.

FIG. 12. Comparative analysis of PDK4 expression between epithelial cells before and after chemotherapy. Each dot represents an individual patient, with the data of “before” and “after” connected to allow direct evaluation of PDK4 induction in a same patient. Left, epithelial cells; Right, stromal cells.

FIG. 13. Pathological correlation of PDK4, CXCL8, WNT16B, p16INK4a and p21CIP1 in the stroma of PCa patients after treatment. Scores from assessment of molecule-specific IHC staining, with expression levels colored to reflect modest (turquoise) via low (blue) and fair (yellow) to high (red) signal intensity. Columns represent individual patients, while rows represent different factors. A total of 48 patients who underwent chemotherapy were analyzed, and the average score for each patient was derived from three independent pathological readings.

FIG. 14. Kaplan-Meier analysis of PCa patients. Disease-free survival (DFS) stratified according to PDK4 expression (low, average score <2, n=22; high, average score >2, n=26). DFS represents length (months) of period calculated from date of PCa diagnosis to point of first time disease relapse. Survival curve was generated by Kaplan-Meier method. HR, hazard ratio.

FIG. 15. Representative images of PDK4 expression in biospecimens of human breast cancer (BCa) patients after histological examination. Left, untreated, wherein the left images are the results of immunohistochemical (IHC) staining and the right images are the results of hematoxylin-eosin (HE) staining; right, chemo-treated, wherein the left images are the results of immunohistochemical (IHC) staining and the right images are the results of hematoxylin-eosin (HE) staining. The right figure shows the staining results of hematoxylin and eosin (HE). The rectangular region selected in the upper image of each staining is magnified in the lower image. Scale bar, 100 μm.

FIG. 16. Pathological evaluation of stromal PDK4 expression in BCa samples (untreated, 68 cases; treated, 62 cases). Patients were classified into four categories based on the IHC staining intensity of PDK4 in the stroma. 1, negative; 2, weak; 3, moderate; 4, strong expression. Left, statistical comparison. Right, representative images of PDK4 siginals in each category. EL, expression level. Scale bar, 100 μm.

FIG. 17. Boxplot summary of PDK4 transcript expression by qRT-PCR analysis upon laser capture microdissection (LCM) of cells from tumor and stroma, respectively. Signals normalized to the lowest value in untreated epithelium group, with comparison performed between untreated (68 cases) and treated (62 cases) samples per cell lineage. For epithelial or stromal-derived cells, samples from 10 untreated and treated patients in each group were randomly selected for further analysis and parallel comparison.

FIG. 18. Kaplan-Meier analysis of BCa patients. Disease-free survival (DFS) stratified according to PDK4 expression (low, average score <2, n=27; high, average score >2, n=35). DFS represents length (months) of period calculated from date of BCa diagnosis to point of first time disease relapse. Survival curve was generated by Kaplan-Meier method. HR, hazard ratio.

FIG. 19. A schematic molecular roadmap briefly outlining the landscape of glucose metabolism in mammalian cells.

FIG. 20. Partial metabolic profiling (glycolysis) of senescent cells induced by BLEO and incubated with uniformly labeled [U-13C6]-glucose. Results from gas chromatography-mass spectrometry (GC-MS) analysis of metabolites, including DHAP, GAP, 3PG, pyruvate and lactate, as indicated.

FIG. 21. Partial metabolic profiling (TCA cycle) of senescent cells induced by TIS and incubated with [U-13C6]-glucose. The disclosure presents the results from GC-MS analysis of metabolites, including citrate, α-ketoglutarate, succinate, fumarate, malate and so on. TCA, tricarboxylic acid cycle.

FIG. 22. Heatmap depicting changes of glucose catabolism-associated metabolites as measured for senescent cells by GC-MS.

FIG. 23. Statistic comparison of citrate M2 and pyruvate M3 in control and senescent cells induced by BLEO treatment.

FIG. 24. Representative TEM images showing the ultrastructural profile of mitochondria in human stromal cells. The rectangular region selected in the left image of each staining is magnified in the right image. Scale bars in the left, 1 μm. Scale bars in the right, 1 μm. L, low resolution; H, high resolution.

FIG. 25. Measurement of extracellular fluids with an XF24 Extracellular Flux Analyzer.

FIG. 26. OCR of stromal cells was measured using an XF24 Extracellular Flux Analyzer. In simple, 1.5 μM oligomycin, 0.5 μM FCCP and 0.5 μM Rot/Ant were injected sequentially in order into each well. The glycolysis rate was calculated as the difference between the maximum rate measured before injection and the last rate measured before glucose injection. All Seahorse data were normalized to cell numbers, with all metabolic parameters automatically calculated by WAVE software equipped in the Seahorse. OCR, oxygen consumption rate. Oligo, oligomycin. FCCP, carbonyl cyanide 4-(trifluoromethoxy) phenylhydrazone. Rot, rotenone. Ant, antimycin. IN, PDK4-IN (PDK4 inhibitor, 10 nM).

FIG. 27. Measurement of the glycolytic capacity (the maximum ECAR rate in cells after adding oligomycin) of stromal cells. Data derived from ECAR assays and presented in mpH/min.

FIG. 28. Assessment of the glycolytic reserve (the proximity extent between the abilities of cellular response to energy demands and cellular glycolytic functions and the theoretical maximum value) of stromal cells. Data derived from ECAR assays and presented in mpH/min.

FIG. 29. Extracellular acidification rate profile of stromal cells was determined using a Glycolysis Stress Test kit.

FIG. 30. Measurement of ATP production by stromal cells. ATP production measured as (last rate measurement before Oligo injection) minus (minimum rate measurement after Oligo injection).

FIG. 31. Assessment of basal respiration as an essential element of the senescence-associated metabolism program.

FIG. 32. Examination of maximal respiration as another fundamental element of the senescence-associated metabolism program.

FIG. 33. Assessment of non-mitochondrial oxygen consumption in stromal cells.

FIG. 34. Measurement of pH values in stromal cells.

FIG. 35. Determination of lactate production in stromal cells.

FIG. 36. Examination of the leak of H⁺ (proton) from mitochondria of stromal cells.

FIG. 37. Quantitative RT-PCR to examine expression of glucose uptake and metabolism-associated genes after PSC27 cells were subject to individual treatment. GLUT1: glucose transporter 1; HK1: hexokinase 1; HK2: hexokinase 2; LDHA: lactate dehydrogenase A; PFK1: Phosphofructokinase 1; PKM2: Pyruvate Kinase M2; IDH2, isocitrate dehydrogenase 2; IDH3, isocitrate dehydrogenase 3; OGDH, oxoglutarate dehydrogenase; CS: citrate synthase.

FIG. 38. Evaluation of cellular senescence of PSC27 by SA-β-Gal staining. Left, comparative statistics. Middle, representative images. Scale bar, 100 μm. Right, immunoblot analysis of PDK4, CXCL8, IL6 expression in stromal cells expressing exogenous PDK4.

FIG. 39. Glucose uptake measurement of PSC27 cells upon senescence after drug induction. CTRL, control; DTX, docetaxel; PTX, paclitaxel; VBL, vinblastine; BLEO, bleomycin; DOXO, doxorubicin; MIT, mitoxantrone.

FIG. 40. Examination of pH values of human stromal cells treated similarly in FIG. 39.

FIG. 41. Glucose uptake measurement of PSC27 cells transduced with a PDK4 construct or depleted of PDK4 via shRNA. OE, over expression. SCR, scramble control.

FIG. 42. Lactate production assessment of PSC27 sublines as described in FIG. 41.

FIG. 43. Relative TG production assay of PSC27 sublines as described in FIG. 41.

FIG. 44. Determination of the pH of conditioned media of PSC27 sublines as described in FIG. 41.

FIG. 45. Glucose uptake measurement of PSC27 cells upon BLEO-induced senescence (TIS) in the presence or absence of PDK4, the latter mediated by shRNA knockdown.

FIG. 46. Lactate production assessment of PSC27 cells as described in FIG. 45.

FIG. 47. Relative TG production assay of PSC27 cells as described in FIG. 45.

FIG. 48. Determination of the pH of conditioned media of PSC27 cells as described in FIG. 45.

FIG. 49. Comparative results of PDK4, CXCL8, glycolysis-related genes (Glut1, MCT4, HIF1α, PGK1 and PGI) as well as and TCA-related genes (CS, IDH2, IDH3A and IDH3B) expression in human stromal cells 7 d after treatments.

FIG. 50-52. Heatmap depicting differentially expressed human transcripts in PC3 (FIG. 50), DU145 (FIG. 51), M12 (FIG. 52) and other PCa lines after a 3-d culture with the CM of PSC27 cells overexpressing PDK4 (PSC27-PDK4). In contrast to cancer cells cultured with control CM (PSC27-CTRL), the number of genes up- and down-regulated per PCa line are indicated. Intensity of tracing lines consistent with the relative expression fold change averaged per up- or down-regulated genes.

FIG. 53-55. Graphical visualization by pathway analysis (pie chart depicting biological processes). Genes significantly enriched were sorted according to fold change in PC3 (FIG. 53), DU145 (FIG. 54), M12 (FIG. 55) and other PCa lines exposed to the CM of PSC27-PDK4 cells.

FIG. 56. Venn diagram displaying the overlap of transcripts co-upregulated in PC3, DU145 and M12 cells (per 2 or 3 lines) upon treatment with the CM from PSC27-PDK4 in contrast to those treated with the CM of PSC27-CTRL.

FIG. 57. Summary of transcripts co-upregulated in PC3, DU145 and M12 cell lines (top ranked, with FC>5.0 and FDR<0.01) upon treatment with the CM of PSC27-PDK4. Red highlight indicates HTR2B.

FIG. 58. Immunoblot measurement of HRT2B protein expression in 3 PCa cell lines (PC3, DU145, M12). Glyceraldehyde-3-phosphate dehydrogenase (GAPDH), protein loading control.

FIG. 59. Left, appraisal of the proliferation capacity of PCa lines under different conditions. As the experimental group, human HTR2B was transduced into each PCa cell line, and LY 266097 was used in the culture medium as a control for drug treatment. Middle, measurement of the migration ability of PCa lines under different conditions, with cells treated similarly as described above. Right, measurement of the migration ability of PCa lines under different conditions, with cells treated similarly as described above.

FIG. 60. Determination of the resistance of PCa lines to MIT upon exposure to the CM of PSC27-PDK4 stromal cells. MIT, mitoxantrone, a chemotherapeutic agent applied at the IC50 concentration per cell line established prior to the assay.

FIG. 61. Dose-response curves (non-linear regression/curve fit) plotted from MIT-based viability assays of PC3 exposed to the CM of PSC27-PDK4 cells and treated by a range of concentrations of MIT.

FIG. 62. Statistical analysis of tumor end volumes. PC3 xenografted alone or together with PSC27 to the hind flank of animals and tumor volumes were measured at the end of the 8-week experimental cycle.

FIG. 63. Schematic workflow of experimental procedure in mice with severe combined immunodeficiency (SCID). Two weeks after the subcutaneous implantation and in vivo uptake of tissue recombinants, animals were received single or combined drugs as rhythmic therapy consisting of several cycles.

FIG. 64. Two weeks before chemotherapy, cancer cells (PC3) alone or combined with stromal cells (PSC27) were inoculated subcutaneously to NOD/SCID male mice. Agents delivered on 1 st day of each week starting from 3rd week, then given every other week, totally 3 doses. At the end of 8 weeks, mice were sacrificed, with tumors be measured and tissues be assessed.

FIG. 65. Statistical analysis of tumor end volumes. PC3 cells xenografted alone or together with PSC27 cells to the hind flank of SCID mice. MIT and PDK4-IN administered either alone or concurrently to induce tumor regression. Right, representative tumor images.

FIG. 66. Transcript assessment of several canonical SASP factors expressed in stromal cells isolated from tumors in SCID mice. LCM isolation, total RNA preparation and expression assays on tissues from animals with simultaneously implanted stromal cells and cancer cells. Note that the group measured as of the lowest value was used as normalization baseline per factor.

FIG. 67. Transcript assessment of individual canonical SASP factors (AREG), aging markers (p16/p21) and PDK4 expressed in stromal cells isolated from tumors in SCID mice. LCM isolation, total RNA preparation and expression assays on tissues from animals with simultaneously implanted stromal cells and cancer cells. Note that the group measured as of the lowest value was used as normalization baseline per factor.

FIG. 68. Representative IHC images of SA-β-Gal staining chromatograms from tissues isolated from animals subjected to placebo or drug treatments. Scale bar, 100 μm. Right, Comparative statistical analysis of SA-β-Gal staining for tissues in mice mentioned above.

FIG. 69. Statistical evaluating values of DNA-damaged cells and apoptotic cells in analyzed tumor specimens, represented as a percentage of cells positively stained by IHC with antibodies against γ-H2AX or caspase 3 (cleaved).

FIG. 70. Representative IHC images of caspase 3 (cleaved) in tumors at the end of therapeutic regimens. Biopsies of placebo-treated animals served as negative controls for drug-treated mice. Scale bar, 100 μm.

FIG. 71. Statistical analysis of tumor end volumes. MDA-MB-231 breast cancer cells xenografted alone or together with HBF1203 breast stromal cells to the hind flank of SCID mice. DOX and PDK4-IN administered either alone or concurrently to induce tumor regression. MDA, MDA-MB-231. DOX, doxorubicin.

FIG. 72. Statistical analysis of tumor end volumes. PC3 cells xenografted alone or together with PSC27 cells to the hind flank of SCID mice. PTX and PDK4-IN administered either alone or concurrently to induce tumor regression. PTX, paclitaxel.

FIG. 73. Statistical analysis of tumor end volumes. MDA-MB-231 cells xenografted alone or together with HBF1203 cells to the hind flank of SCID mice. DTX and PDK4-IN administered either alone or concurrently to induce tumor regression. MDA, MDA-MB-231. DTX, docetaxel.

FIG. 74. Curve of bulky DFS plotted against survival time of implantation with recombinant tissues in mice until animal death attributed to advanced bulky disease development. MS, median survival. P values calculated by two-sided log-rank (Mantel-Cox) tests.

FIG. 75. Measurement of lactate concentration in serum of untreated and chemotherapy-treated PCa patients. Data were derived from ELISA measurement and are shown as mean±SD. n=20.

FIG. 76. ELISA analysis of CXCL8 protein abundance in the serum of patients analyzed above and results were described as mean±SD. n=20.

FIG. 77. ELISA analysis of SPINK1 protein abundance in the serum of patients analyzed above and the results were described as mean±SD. n=20.

FIG. 78. Scatter-plot showing correlation between lactate and CXCL8 in the serum of individual patients described above, with Pearson's correlation coefficient, P value and confidence interval indicated.

FIG. 79. Scatter-plot showing correlation between lactate and SPINK1 in the serum of individual patients described above, with Pearson's correlation coefficient, P value and confidence interval indicated.

FIG. 80. Heat-map depicting overall correlation between stromal CXCL8, serum CXCL8, stromal SPINK1 and serum SPINK1 in chemo-treated patients. Raw scores of stromal CXCL8 and SPINK1 from independent pathological reading of primary tumors of PCa patients, with those raw scores of serum CXCL8 and SPINK1 from ELISA detection. Color key, relative expression of these factors in stroma or serum of patients.

FIG. 81. Kaplan-Meier survival analysis of chemo-treated patients with PCa. Disease-free survival (DFS) stratified according to cycling lactate in serum (low, average score <2, dark green; high, average score >2, dark red). DFS represents length (months) of period calculated from the date of chemotherapy to point of first time disease relapse. Survival curves generated according to the Kaplan-Meier method, with a P value calculated using a log-rank (mantel-cox) test. n=10 per group.

FIG. 82. TCGA data showing alterations of PDK4 in a variety of human cancer types at genomic level, including mutation, amplification and deep deletion. Alteration frequency displayed in percentage. CNA represents tumor copy number alteration.

FIG. 83. Schematic design of 20-month-old C57BL/6J mice undergoing either placebo or PDK4-IN intervention.

FIG. 84. Comparison and analysis of several livers in mice after SA-β-gal staining and HE staining.

FIG. 85. Statistical analysis of senescent cells in the microenvironment of key organs in mice after SA-β-Gal and H&E staining. Liver (left 1st), lung (left 2nd), prostrate (left 3rd) and myocardium (left 4th).

FIG. 86. Quantification of alveolar size in mice after HE staining. Left, representative images. Right, statistical comparative analysis.

FIG. 87-88. Quantitative measurement of experimental mice described above by a series of physical function tests, including maximal walking speed, hanging endurance, performance time of rotarod (FIG. 84), grip strength, treadmill endurance, daily activity (FIG. 85), and so on.

FIG. 89. The serum levels of alanine transaminase (ALT), aspartate transaminase (AST) and lactate dehydrogenase (LDH) of serum in each group of mice.

FIG. 90. Quantitative results of body weights and food intake in experimental mice described above.

FIG. 91. Schematic design for lifespan appraisal of mice. Mice aged 24 to 27 months were administered either Vehicle or PDK4-IN every two weeks, with continuous monitoring of their survival and recording of maximum lifespan.

FIG. 92 shows the post-treatment survival curves for mice in preclinical stage. C57BL/6 mice aged 24 to 27 months were administered either Vehicle or PDK4-IN every two weeks intraperitoneally. The median survival of animals in each group was calculated and indicated. ***, P<0.001.

### DETAILED DESCRIPTION

After in-depth researches, the inventors revealed pyruvate dehydrogenase kinase 4 (PDK4) as a novel target for intervention of cellular senescence and intervention in drug resistance of tumor. By targeting PDK4, drugs can be developed to inhibit drug resistance of tumor, suppress or eliminate the SASP. Additionally, drugs targeting PDK4 can be used in combination with chemotherapeutic drugs for diagnosis or prognosis of related diseases or symptoms based on the relevant target and mechanism.

The inventors discovered that, despite chemotherapeutic drugs are able to inhibit cancer cells, they have a significant impact on the tumor microenvironment, leading to notable side effects, especially enhancing the metabolism of senescent cells, also with induced drug resistance in cancer cells after using for a long time. Although PDK4 neither induces nor affects cellular senescence and SASP factors (SASP), surprisingly, the inhibitor of PDK4 (PDK4-IN), when used in combination with specific chemotherapeutic drugs, effectively suppresses the hypermetabolic phenotype of senescent cells, exerting a beneficially synergistic and unexpectedly enhanced effect.

Senescent cells and metabolism Normal differentiated cells primarily rely on mitochondrial oxidative phosphorylation for cellular energy supply, while most cancer cells depend on aerobic glycolysis. This phenomenon is termed as “Warburg effect.” Warburg observed that unlike most normal tissues, cancer cells favor glucose “fermentation” into lactate even in the presence of sufficient oxygen to support mitochondrial oxidative phosphorylation (Vander Heiden M G, Cantley L C, Thompson C B. Understanding the Warburg effect: the metabolic requirements of cell proliferation. Science. 2009; 324(5930):1029-1033. doi:10.1126/science. 1160809). Senescent cells also alter their metabolism in a way similar to aerobic glycolysis.

Glucose is the primary carbon source to aerobic glycolysis, followed by glutamate and aspartate (non-protonatable amino acids as glutamine or asparagine, respectively) as secondary sources. FIG. 19 shows a schematic molecular roadmap briefly outlining the landscape of glucose metabolism in mammalian cells. In mammalian cells, the intermediate metabolite of glucose, pyruvate, enters the tricarboxylic acid (TCA) cycle through pyruvate dehydrogenase complex (PDH), while the pyruvate dehydrogenase kinase (PDK) molecule (PDK1-4) inhibits PDH activity and promotes the switching of metabolic pathways from mitochondrial oxidation to cytoplasmic glycolysis.

Pyruvate is the final metabolite of glycolysis. Pyruvate can be metabolized into lactate or transferred to the mitochondria and metabolized into acetyl coenzyme A (acetyl-CoA), which is essential for the synthesis of citric acid in the tricarboxylic acid (TCA) cycle. The TCA cycle involves the condensation of acetyl-CoA with oxaloacetate to form citrate. Subsequently, the formed citrate is decomposed through a series of enzymatic steps to generate additional ATP molecules. In the processes of glycolysis and the TCA cycle, various metabolites are produced, including but not limited to: dihydroxyacetone phosphate (DHAP), 3-phosphoglyceraldehyde (GAP), pyruvate, alanine, lactate, 3-phosphoglyceric acid (3-PG), citrate, α-ketoglutarate, glutamate, succinate, succinate, fumarate and malate. As used herein, molecules and enzymes associated with glucose uptake and metabolism include but are not limited to: glucose transporter 1 (GLUT1), hexokinase 2 (HK2), lactate dehydrogenase A (LDHA), isocitrate dehydrogenase 2 (IDH2), isocitrate dehydrogenase 3 (IDH3), oxoglutarate dehydrogenase (OGDH), citrate synthase (CS). Therefore, as used herein, the ability of glycolysis and/or the activity of TCA cycle can be reflected by indicators including citrate/pyruvate ratio, and/or levels of metabolites, and/or enzyme expression or activity in metabolic processes, and/or oxygen consumption rate (OCR), and/or extracellular acidification rate (ECAR), and/or ATP production, and/or basal respiration, and/or basal respiration, and/or maximal respiration, and/or non-mitochondrial oxygen consumption, and/or pH value (of extracellular fluid), and/or lactate production, and/or proton leak.

As used herein, senescent cells remain a “hypermetabolic phenotype”, reflected at the molecular level by an increased citrate/pyruvate ratio, elevated levels of metabolites, and increased expression or activity of enzymes in metabolic processes, perturbations in mitochondrial ultrastructure, significantly increased extracellular acetone and lactate production and extracellular acidification rate (ECAR), increased oxygen consumption rate (OCR), and elevated ATP production, basal respiration and maximal respiration.

Pyruvate dehydrogenase (PDH) complex is a crucial component in the process of glucose metabolism, catalyzing the conversion of pyruvate to acetyl-CoA and serving as an intersection between glycolysis and oxidative phosphorylation pathways. The activity of the PDH complex depends on its phosphorylation status. Pyruvate dehydrogenase kinases (PDKs) and pyruvate dehydrogenase phosphatases (PDPs) play significant roles in regulating the phosphorylation levels and activity of the PDH complex. There are four different pyruvate dehydrogenase kinases (PDK1-4) in human, each playing a distinct role in regulating enzyme activity in response to different environmental stimuli. Among the four enzymes, PDK4 predominantly controls the activity of the PDH complex. Located in the mitochondrial matrix, PDK4 inhibits the PDH complex by phosphorylating its E1α subunit, thereby promoting the regulation of glucose metabolism. PDK4 is mainly expressed in muscles (hearts and skeletal) and influences the metabolic fate of glucose during exercise.

As used herein, PDK4 is a key enzyme involved in regulating glucose and fatty acid metabolism as well as tissue homeostasis. PDK4 overexpression deactivates the PDH complex by phosphorylating specific targets. In specific embodiments, when a down-regulator of PDK4 or an encoding gene thereof (for example, PDK4-IN) are used in senescent cells, changes of non-mitochondrial oxygen consumption, pH fluctuations, lactate production and proton leak are reversed, also with alterations in the expression of molecules and enzymes associated with glucose uptake and metabolism simultaneously.

### PDK4

In the present disclosure, the term “PDK4” refers to the protein with GenBank accession number ID 5166 (human).

The term “PDK4” also comprises variants of the sequence with the same function as PDK4. The variants include but are not limited to: deletion, insertion and/or substitution of one or more (usually 1-50, preferably 1-30, more preferably 1-20, most preferably 1-10, even more preferably 1-8, 1-5) amino acids, and addition or deletion of one or several (usually within 20, preferably within 10, more preferably within 5) amino acids at the C-terminal and/or N-terminal. For example, in the art, substitution with amino acids of approaching or similar property generally does not alter the function of a protein. For another example, the addition of one or several amino acids to C-terminal and/or N-terminal also generally does not alter the function of a protein. The term also comprises functional fragments and functional derivatives of PDK4.

Above term “PDK4” also comprises proteins that are (80% or more, more preferably 85% or more, such as 90%, 95%, 98% or 99%) homologous to the polypeptide sequence with the GenBank accession number described above, with substantially the same functions of the PDK4 described in examples in the present disclosure. These proteins are also included in the present disclosure. Methods and tools for aligning sequence identity are also well known in the art, such as BLAST. “Homology” refers to the similarity (i.e., sequence similarity or identity) between two or more nucleic acids or polypeptides in terms of certain percentage of amino acid residues in the same positions.

Nucleotide sequences (encoding sequences) encoding PDK4 or conserved variant proteins thereof can also be applied in the present disclosure. The term “encoding gene” can include a polynucleotide encoding the protein, or a polynucleotide that further includes additional coding and/or non-coding sequences.

In researches of the inventors, it was found that PDK4 is highly expressed in senescent cells induced by genotoxic (DNA-damaging) stress. Additionally, PDK4 is highly expressed in tissues of clinical cancer patients after new adjuvant chemotherapy with genotoxic drugs like mitoxantrone (MIT). The high expression of PDK4 is associated with a (negative) correlation with disease-free survival (DFS). PDK4 positive senescent cells alter the expression profile and malignant phenotype of cancer cells through 5-hydroxytryptamine receptor 2B (HTR2B), but PDK4 per se neither influenced cell senescence, nor affected the expression level of typical SASP factors.

The inventors also discovered that senescent cells develop a distinctive hypermetabolic phenotype characterized of enhanced glycolysis, TCA cycle activity and ATP-boosting oxidative phosphorylation. Besides, senescent cells produce lactate via glucose consumption by PDK4 high expression.

Furthermore, by animal experiments, the inventors found that selective targeting of PDK4 can effectively inhibit the hypermetabolic phenotype in senescent cells induced by chemotherapeutic drugs, preventing drug resistance of chemotherapy and significantly improving overall efficacy. Classic chemotherapy combined with PDK4 targeting inhibitors can induce tumor regression, and its indicators are significantly better than those caused by chemotherapy alone. The disease-free survival period of animals receiving combined treatment with chemotherapy drugs+PDK4 inhibitors is significantly prolonged.

Additionally, the inventors found that selectively targeting PDK4 in mice with natural senescence can reduce serum lactate levels, decrease the number of senescent cells in tissues, and extend the median survival period, thus postponing aging.

These results reveal that PDK4 plays a critical role in both normal and pathological states and can serve as a novel target for intervention in cellular senescence and intervention in drug resistance of tumor.

### PDK4 Down-Regulators and Uses Thereof

Based on the above new discoveries by the inventors, the present disclosure provides a use of a down-regulator of PDK4 or an encoding gene thereof, for preparing a pharmaceutical combination for inhibiting drug resistance of tumor or inhibiting/eliminating the SASP.

In the present disclosure, unless otherwise specified, the “tumor” is a tumor with drug resistance. Preferably, the tumor comprises a tumor with drug resistance after exposure to genotoxic drugs or exhibiting the SASP in the tumor microenvironment; more preferably, comprising: prostate cancer, breast cancer, lung cancer, colorectal cancer, gastric cancer, liver cancer, pancreatic cancer, bladder cancer, skin cancer, kidney cancer.

In some embodiments of the present disclosure, the “SASP” is a SASP caused by DNA damage; preferably, the DNA damage is a DNA damage caused by a chemotherapeutic drug; more preferably, the chemotherapeutic drug comprises a genotoxic drug. Specifically, in some embodiments of the present disclosure, the “SASP” is a “hypermetabolic phenotype”, a distinctive phenotype characterized of enhanced glycolysis, TCA cycle activity, ATP-boosting oxidative phosphorylation and increased energy production.

As used herein, the down-regulator of PDK4 or an encoding gene thereof comprises inhibitors, antagonists, blockers, blocking agents, and similar terms, which can be used interchangeably.

The down-regulator of PDK4 or an encoding gene thereof refer to any substance that can decrease the activity of PDK4, decrease the stability of PDK4 or the encoded protein thereof, down-regulate the expression of PDK4, shorten the effective time of PDK4, or inhibit the transcription and translation of PDK4 gene. These substances can be used in the present disclosure as useful substances for down-regulating PDK4. For example, the down-regulators are: interfering RNAs or antisense nucleotides that specifically interfering gene expression of PDK4; or an antibody or a ligand that specifically bind to the protein encoded by the PDK4 gene, and so on.

As a selected embodiment of the present disclosure, the down-regulator may be a small-molecule compound targeting PDK4. Conventional screening methods can be used by those skilled in the art for screening such small-molecule compounds. For example, in preferred embodiments of the present disclosure, an optional screening method is provided.

In a preferred embodiment of the present disclosure, the down-regulator is PDK4-IN. The term “PDK4-IN” refers to an anthraquinone derivative, a PDK4-specific inhibitor, with CAS number 2310262-10-1.

Those skilled in the art should understand that, after knowing the structure of the PDK4-IN of the present disclosure, the compound of the present disclosure can be obtained by various methods well known in the art, by using known materials in the art, such as methods of chemical synthesis or extraction from organisms (e.g., microorganisms), these methods are all included in the present disclosure. In addition, PDK4-IN is also a commercial drug, so a finished product thereof is easily available to those skilled in the art. The present disclosure also comprises a pharmaceutically acceptable salt, ester, isomer, solvate or prodrug of the PDK4-IN, as long as they retain the same or substantially same functions with the PDK4-IN.

In specific embodiments of the present disclosure, animal experiments show that when the chemotherapeutic drug MIT is combined with PDK4-IN for treatment, PDK4-IN can significantly inhibit drug resistance of tumors, thereby promoting the effectiveness of the chemotherapeutic drug in suppressing tumors, significantly improving animal survival rates. At the same time, in specific embodiments of the present disclosure, the use of PDK4-IN inhibits the hypermetabolic phenotype of cancer cells.

Under specific conditions, usage frequency of down-regulators of PDK4 or an encoding gene thereof may depend on the accumulation rate of senescent cells. However, the accumulation rate of senescent cells may vary depending on the environment in which cellular senescence occurs. For example, repeated exposure to DNA-damaging cancer therapies may lead to a more rapid re-accumulation of senescent cells compared to natural aging. Intermittent use of down-regulators of PDK4 or an encoding gene thereof can reduce the risk of adverse reactions in patients and allow for the use of down-regulators of PDK4 or an encoding gene thereof during healthy periods. In addition, intermittent administration can reduce the side effects of down-regulators of PDK4 or an encoding gene thereof and reduce the likelihood of patients developing drug resistance. Contrary to the situation with anticancer drugs or antibiotics, because senescent cells do not undergo division, the body cannot rely on cell proliferation to generate resistance of down-regulators of PDK4 or an encoding gene thereof and thus cannot acquire favorable mutations. This creates a favorable foundation for the widespread clinical use of down-regulators of PDK4 or an encoding gene thereof.

As a preferred embodiment of the present disclosure, the down-regulator may be PDK4-specific short hairpin RNA (shRNA). Those skilled in the art can understand that, based on the gene sequence of PDK4 in the present disclosure, such shRNA molecules can be prepared. The methods for preparing shRNA molecules are not particularly restrained and include, but are not limited to: chemical synthesis, in vitro transcription, and other methods. The shRNA can be delivered into cells using suitable transfection reagents or other known techniques in the field.

As an optional embodiment in the present disclosure, the down-regulator can be a proteasome or an upregulator of the proteasome.

As an optional embodiment in the present disclosure, the CRISPR/Cas system (such as Cas9) can be employed for targeted gene editing to knockout the PDK4 gene in the region associated with the disease. Common methods for knocking out the PDK4 gene include co-transfecting nucleic acids, such as sgRNA or nucleic acids capable of forming sgRNA, and Cas9 mRNA or nucleic acids capable of forming Cas9 mRNA, into the targeted region or cells. After determining the target site, known methods can be used to introduce sgRNA and Cas9 into the cells. The nucleic acids capable of forming sgRNA or Cas9 mRNA may be nucleic acid constructs or expression vectors, and these expression vectors can be introduced into the cells to form active sgRNA and Cas9 mRNA.

The mentioned approaches represent some typical methods for downregulating PDK4. It should be understood that, once those skilled in the art comprehend the overall scheme of the present disclosure, other known methods in the field can also be employed to regulate PDK4, and these methods are also encompassed by the present disclosure.

### Pharmaceutical Composition

The present disclosure also provides a pharmaceutical composition, wherein it comprises effective amounts (for example, 0.000001-50 wt %; preferably 0.00001-20 wt %; more preferably 0.0001-10 wt %) of the down-regulator of PDK4 or an encoding gene thereof, and a pharmaceutically acceptable carrier.

As a preferred embodiment, the present disclosure provides a composition for inhibiting drug resistance of tumor or inhibiting or eliminating the SASP, wherein the composition comprises an effective amount of a down-regulator of PDK4 or an encoding gene thereof, and a pharmaceutically acceptable carrier.

As used herein, the term “effective amount” refers to an amount that is functional or active for humans and/or animals and is acceptable for administration to humans and/or animals. The term “pharmaceutically acceptable carrier” refers to a carrier for the administration of a therapeutic agent, comprising various excipients and diluents. The term refers to pharmaceutical carriers that are, by themselves, not essential active ingredients and are not unduly toxic after administration. Suitable carriers are well known to those of ordinary skill in the art. Pharmaceutically acceptable carriers in compositions may comprise liquids such as water, saline, buffers. In addition, auxiliary substances such as fillers, lubricants, glidants, wetting or emulsifying agents, pH buffering substances and the like may also be present in these carriers. The carrier may also comprise cell transfection reagents.

After knowing the uses of the down-regulator of PDK4 or an encoding gene thereof, various well-known methods in the field can be employed to administer the down-regulator, the encoding gene, or the pharmaceutical composition to mammals or humans.

Preferably, gene therapy can be used. For example, the down-regulator of PDK4 can be directly administered to the subject through methods like injection. Alternatively, carriers expressing the down-regulator of PDK4 (such as expression vectors or viruses, or shRNA, siRNA) can be delivered to the target, allowing active expression of PDK4 down-regulator. The specific method depends on the type of down-regulators and is well-known to those skilled in the art.

The pharmaceutical composition or mixture of the present disclosure can be prepared into any conventional preparation form by conventional methods. Dosage forms can be diverse, the dosage form is acceptable as long as the active ingredients can effectively reach the body of the mammal. For example, it can be selected from: injection, infusion, tablet, capsule, pill. Wherein, the active components (e.g., PDK4-IN and an optional genotoxic drug) can exist in a suitable solid or liquid carrier or diluent. The mixture of active ingredients or the pharmaceutical composition of the present disclosure may also be stored in a sterile device suitable for injection or infusion. The effective doses of the active ingredients in the composition (e.g., PDK4-IN and an optional genotoxic drug) may vary with the administration mode and the severity of the disease to be treated, which can be based on the experience and recommendations of clinicians.

The down-regulator of PDK4 or an encoding gene thereof and an optional genotoxic drug or the pharmaceutical composition can be administered orally, intravenously, intramuscularly or subcutaneously, etc. Oral administration may be preferred. Pharmaceutical forms suitable for oral administration include, but are not limited to, tablet, powder, capsule, sustained-release formulation, and the like. The pharmaceutical forms suitable for injection include: sterile aqueous solution or dispersion and sterile powder. In all cases, these forms must be sterile and must be fluid to easily drain from syringe. When necessary, the down-regulator of PDK4 or an encoding gene thereof (for example PDK4-IN) and the optional genotoxic drug can also be administered in combination with an additional active ingredient or drug.

The effective amount of the down-regulator of PDK4 or an encoding gene thereof in the present disclosure may vary with the route of administration, the severity of the disease to be treated, and the like. Selection of preferred effective amount can be determined by those skilled in the art based on various factors (e.g. through clinical trials). Such factors comprise but are not limited to: pharmacokinetic parameters (such as bioavailability, metabolism, half-life, and so on) of the down-regulator of PDK4 or an encoding gene thereof, the severity of the disease to be treated, weight, immune status of patients, the route of administration, and so on.

In the specific examples of the present disclosure, some dosing regimens for animals such as mice are given. It is easy for those skilled in the art to convert the dosage of animals such as mice into dosages suitable for humans. For example, it can be calculated according to the Meeh-Rubner formula: A=k′(W2/3)/10,000. In the formula, A is the body surface area, calculated in m²; W is the body weight, calculated in g; K is a constant, which varies with animal species. Generally speaking, mice and rats are 9.1, guinea pigs are 9.8, rabbits are 10.1, cats are 9.9, dogs are 11.2, monkeys are 11.8, human is 10.6. It will be understood that, depending on the drug and the clinical situation, the conversion of the administered dose may vary according to the assessment of an experienced pharmacist.

The present disclosure also provides a pharmaceutical composition comprising the described components or a drug kit comprising the down-regulator of PDK4 or an encoding gene thereof directly. In addition, the kit may also comprise instructions for using the medicine in the kit.

### Uses Related to Diagnosis and Prognostic Assessment

The present disclosure provides a target for crucially regulating drug resistance of tumor or the hypermetabolic phenotype of tumors. Based on this novel discovery by the inventors, PDK4 or its metabolite lactate can serve as a target (or marker) for inhibiting drug resistance of tumor or screening drugs that inhibit drug resistance of tumor, comprising: (i) tumor typing, discrimination, and/or susceptibility analysis; (ii) evaluating therapeutic drugs, drug efficacy, prognosis in relevant populations with drug resistance of tumor, and select appropriate treating methods. For example, a population with abnormal expression of the PDK4 gene can be isolated, allowing for more targeted treatment.

By assessing the expression or activity of PDK4 in the sample to be evaluated, or detecting the lactate level in the sample to be evaluated (such as serum), the tumor prognosis of the subject who provides the sample to be evaluated can be predicted, and appropriate drugs can be selected for the treatment. Usually, a threshold can be specified. When the expression of PDK4 or the level of lactate is higher than the specified threshold, it can be considered to treat with a PDK4-inhibiting regimen. The threshold is easily determined by those skilled in the art, for example, by comparing the expression of PDK4 in cells or tissues of normal individuals with the expression of PDK4 in cells or tissues of patients, or by comparing the lactate level in cells or tissues of normal individuals with the lactate level in cells or tissues of patients, the threshold values are obtained.

Various methods known in the art can be used to detect the presence and expression of the PDK4 gene, or to monitor lactate levels, and these methods are all included in the present disclosure. For example, existing methods such as Southern blotting, Western blotting, DNA sequence analysis, PCR, enzyme detection, and other methods can be used, and these methods can be used in combination.

The present disclosure also provides reagents for detecting the presence and expression of PDK4 or an encoding gene thereof in an analyte, or for monitoring lactate levels.

The present disclosure also provides a kit for detecting the presence and expression of PDK4 gene in an analyte, wherein the kit comprises: a primer that specifically amplifies the PDK4 gene; a probe that specifically identifies the PDK4 gene; or an antibody or a ligand that specially bind to the protein encoded by the PDK4 gene. Furthermore, the present disclosure provides a kit for monitoring lactate levels in an analyte, wherein the kit comprises: an enzyme for monitoring lactate levels.

In addition, the kit may also comprise various reagents required for DNA extraction, PCR, hybridization, color development, etc., including but not limited to: extraction solution, amplification solution, hybridization solution, enzyme, control solution, chromogenic liquid, lotion, etc. Additionally, the kit also comprises instructions for use and/or nucleic acid sequence analysis software.

### Drug Screening

After knowing the close correlation between the high expressions or high activities of PDK4 and the drug resistance of tumor, this characteristic can be used for screening drugs to inhibit expressions or activities of PDK4 or an encoding gene thereof. Truly useful drugs for inhibiting drug resistance of tumor or inhibiting/eliminating the SASP (for example hypermetabolic phenotypes) can be identified from the substances.

Therefore, the present disclosure provides a method for screening a potential substance for promoting chemotherapeutic drugs to inhibit tumors, wherein the method comprises: (1) providing a system of tumor microenvironment, wherein the system comprises cancer cells and stromal cells; (2) treating the system of (1) with a chemotherapeutic drug, inducing a SASP in the tumor microenvironment; (3) adding the candidate substance to the system in (2) and observing its effect on the tumor microenvironment system; if the candidate substance specifically targets to clear senescent cells and/or promotes the growth of stromal cells (non-senescent cells) in a tumor microenvironment (increase the PD rate of stromal cells), then the candidate substance is a potential substance that can be used for promoting chemotherapeutic drugs to inhibit tumors. In a more preferred embodiment, in step (2), it also comprises: administering the down-regulator of PDK4 or an encoding gene thereof before, during or after inducing the senescence-related secretory phenotype in the tumor microenvironment; in step (3), it also comprises: if the candidate substance statistically promotes the down-regulator of PDK4 or an encoding gene thereof to clear senescent cells and/or promotes the growth of stromal cells in a tumor microenvironment, then the candidate substance is a potential substance that can be used in combination with the down-regulator of PDK4 or an encoding gene thereof to inhibit tumors.

The present disclosure also provides a method for screening a potential substance for inhibiting the SASP (for example hypermetabolic phenotype), wherein the method comprises: (1) providing a system of stromal cells, inducing the SASP in the system; (2) adding the candidate substance to the system of (1) and observing its effect on the system of stromal cells. If the candidate substance statistically promotes the down-regulator of PDK4 or an encoding gene thereof to inhibit the SASP (for example hypermetabolic phenotype), then the candidate substance is a potential substance that can be used in combination with the down-regulator of PDK4 or an encoding gene thereof to inhibit the SASP (for example hypermetabolic phenotype).

In a preferred embodiment of the present disclosure, when performing screening, a control group may be established to facilitate the observation of indicator changes in the testing group. The control group can be a system without adding the candidate substance, also with other conditions remaining the same of the testing group.

As a preferred embodiment of the present disclosure, the method also comprises: further cell experiments and/or animal experiments on the obtained potential substances, so as to further select and determine substances really useful for inhibiting tumors, reversing drug resistance in tumors or inhibiting/eliminating the SASP.

On the other hand, the present disclosure also provides potential substances obtained by the screening methods for inhibiting tumors, reversing drug resistance in tumors or inhibiting/eliminating the SASP. These preliminary screening substances can constitute a library for screening, so that people can finally screen for really useful drugs.

The disclosure if further illustrated by the specific examples described below. It should be understood that these examples are merely illustrative, and do not limit the scope of the present disclosure. The experimental methods without specifying the specific conditions in the following examples generally used the conventional conditions, such as those described in J. Sambrook, Molecular Cloning: A Laboratory Manual (3^{rd} ed. Science Press) or followed the manufacturer's recommendation.

### First Part: Materials and Methods

### 1. Cell Culture

### (1) Cell Line Maintenance

Primary stromal cell line PSC27 of human prostate origin (obtained from Fred Hutchinson Cancer Research Center in the United States) were cultured and passaged in complete PSCC medium. Epithelial cell line BPH1 of benign prostate origin and epithelial cell lines PC3, DU145, M12, LNCaP, 22RV1 of prostate cancer origin and MDA-MB-231 of breast cancer origin (purchased from ATCC) were all cultured in RPMI-1640 complete medium with 5% FBS at 37° C. in a 5% CO2 humidified incubator.

### (2) Cell Cryopreservation and Recovery

a. Cell Cryopreservation

Cells in the logarithmic growth phase were collected with 0.25% trypsin, centrifuged at 1000 rpm for 2 min. The supernatant was discarded, and the cells were resuspended in freshly prepared freezing solution. The cells were subpackaged into labeled sterile cryovials. Then they were cooled by reducing temperature in gradient manner (4° C. 10 min, −20° C. 30 min, −80° C. 16-18 h), and finally transferred to liquid nitrogen for long-term storage.

b. Cell Recovery

The cells frozen in liquid nitrogen were taken out and immediately placed in a 37° C. water bath to allow them to thaw quickly. Two mL of cell culture medium was added directly to suspend the cells evenly. After the cells adhered to the wall, fresh culture medium was used for replacement.

### (3) In Vitro Experimental Treatment

To induce cellular senescence, PSC27 cells were grown until 80% confluent (referred to as PSC27-Pre) and treated with 100 nM docetaxel (DTX), 100 nM paclitaxel (PTX), 200 nM vinblastine (VBL), 10 μM doxorubicin (DOXO or DOX), 50 μg/ml bleomycin (BLEO), 1 μM mitoxantrone (MIT), or 10 Gy 137Cs ionizing radiation (γ-radiation at 743 rad/min, RAD) to the culture medium. After 6 hours of drug treatment, the cells were simply washed 3 times with PBS, left in the culture medium for 7-˜10 days, and then the subsequent experiments were performed.

### 2. Plasmid Preparation and Lentiviral Transduction

Full-length human PDK4 was cloned into the lentiviral expression vector pLenti-CMV/To-Puro-DEST2 (Invitrogen) between the BamHI and XbaI restriction enzyme sites. Packaging cells 293FT were used for cell transfection and lentivirus production. The sequences for small hairpin RNAs (shRNAs) used for knocking down PDK4 were as follows: caaatagtttccctaatcatc (SEQ ID NO: 1) and ctacgggtgcatcagataatt (SEQ ID NO: 2).

### 3. Immunofluorescence and Histological Analysis

Mouse monoclonal antibody anti-phospho-Histone H2A.X (Ser139) (clone JBW301, Millipore) and mouse monoclonal antibody-anti-PDK4 (Cat #sc-518061, Santa Cruz), and secondary antibody Alexa Fluor®488 (or 594)-conjugated F(ab′)2 were sequentially added to the slides coated with the fixed cells. Nuclei were counterstained with 2 g/ml of 4′, 6-diamidino-2-phenylindole (DAPI). The most representative image was selected from the three observation fields for data analysis and result display. FV1000 laser scanning confocal microscope (Olympus) was used to acquire confocal fluorescent images of cells.

The PDK4 antibody used for immunohistochemistry (IHC) staining in tissues from clinical patients with prostate cancer and breast cancer was same as the PDK4 antibody above, purchased from Santa Cruz. Specific steps are as follows: After routine deparaffinization, the tissues were incubated with 0.6% H₂O₂ in methanol at 37° C. for 30 minutes, followed by antigen retrieval in 0.01 μM pH 6.0 citrate buffer for 20 minutes and cooling at room temperature for 30 minutes. Blocking was performed with normal goat serum for 20 minutes, followed by incubation with the primary PDK4 antibody (1:200) at 37° C. for 1 hour and overnight at 4° C. On the following day, the slides were washed three times with TBS, incubated with the secondary antibody (goat anti-mouse HRP-conjugated) at 37° C. for 45 minutes, washed again three times with TBS, and finally, DAB was used for visualization.

### 4. Co-Culture of Stromal Cells and Epithelial Cells and In Vitro Experiments

After PSC27 cells were cultured in DMEM+0.5% FBS medium for 3 days, then the cell population was washed thoroughly with 1×PBS. After a simple centrifugation, the supernatant was collected as conditioned medium, stored at −80° C. or used directly. Prostate epithelial cells are subjected to in vitro experiments during continuous culture in this conditioned medium for 3 days. For chemotherapy resistance, epithelial cell lines were cultured either in low-serum DMEM (0.5% FBS) (referred to as “DMEM”) or in conditioned medium. Simultaneously, mitoxantrone (MIT) was used to treat cells for 1 to 3 days at concentrations close to the IC50 values for each cell line, followed by observation under a bright-field microscope.

### 5. Whole-Genome Agilent Expression Microarray

The procedures and methods for conducting whole-genome Agilent expression microarray (4×44k) on primary stromal cell line PSC27 of human prostate origin are consistent with those previously published in the paper (Sun, Y., Campisi, J., Higano, C., Beer, T. M., Porter, P., Coleman, I., True, L. and Nelson, P. S. 2012. Treatment-Induced Damage to the Tumor Microenvironment Promotes Prostate Cancer Therapy Resistance through WNT16B. Nat. Med. 18: 1359-1368).

### 6. Whole-Transcriptome Sequencing Analysis (RNA-Sequencing)

Whole-transcriptome sequencing was performed with the primary human prostate stromal cell line PSC27 under different treatment conditions. Total RNA samples were obtained from stromal cells. Their integrity was verified by Bioanalyzer 2100 (Agilent), RNA was sequenced by Illumina HiSeq X10, and gene expression levels were quantified by software package rsem (https://deweylab. github. io/rsem/). Briefly, rRNA was depleted from RNA samples with the RiboMinus Eukaryote Kit (Qiagen, Valencia, CA, USA); and according to the manufacturer's instructions, TruSeq Stranded Total RNA Preparation Kits (Illumina, San Diego, CA, USA) was used to construct a strand-specific RNA-seq library before deep sequencing.

Paired-end transcriptomic reads were mapped to a reference genome (GRCh38/hg38), and reference-annotation was performed from Gencode v27 using the Bowtie tool. Duplicate reads were identified using the picard Tools (1.98) script to mark duplicates (https://github. com/broadinstitute/picard), and only non-duplicate reads were retained. Reference splice junctions were provided by reference transcriptome (Ensembl Build 73). FPKM values were calculated with Cufflinks, and Cufflinks maximum likelihood estimation function was used to call differential gene expression. Genes with significant changes in expression were defined by false discovery rate (FDR)-corrected P-values <0.05, and only ensembl genes 73 with status “Known” and biotype “coding” were used for downstream analysis.

Next, Trim Galore (v0.3.0) (http://www.bioinformatics. babraham. ac. uk/projects/trim_galore/) was used to trim the reads, while the quality assessment was carried out by using FastQC (v0.10.0) (http://www.bioinformatics. bbsrc. ac. uk/projects/fastqc/). Subsequently, by using DAVID bioinformatics platform (https://david.ncifcrf.gov/), Ingenuity Pathways Analysis (IPA) program (http://www.ingenuity.com/index.html), a preliminary analysis of the raw data was perform on a free online platform, Majorbio I-Sanger Cloud Platform (www.i-sanger. com) and the raw data were deposited in the NCBI Gene Expression Omnibus (GEO) database with access code GSE198110.

### 7. Measurement of Gene Expression by Quantitative PCR (RT-PCR)

### (1) Extraction of Total Cellular RNA

Total RNA of cells in the growth phase was extracted with Trizol reagent. 1 ml Trizol was added to each T25 culture flask. The cell layer was scraped off with a cell scraper, transferred into a centrifuge tube and mixed well until not viscous. One fifth mL of chloroform was added for every 1 ml of Trizol, shaken vigorously for 15 seconds, incubated at room temperature for 5-10 min; centrifuged at 11,000 g for 15 min at 4° C.; the colorless supernatant was transferred into a new centrifuge tube, added with 0.5 mL of isopropyl alcohol per 1 ml Trizol, incubated at room temperature for 10 min, centrifuged at 11,000 g and 4° C. for 10 minutes; the supernatant was discarded, washing was performed with 75% ethanol (at least 1 ml 75% ethanol per 1 ml Trizol was used), centrifuged at 4° C. and 7,500 g for 5 minutes; the RNA was precipitated at room temperature for 5-10 minutes (RNA could not be dried), and the precipitate was dissolved with DEPC-H2O.

After the RNA was quantified by a spectrophotometer, a small amount of total RNA was taken for 1% agarose electrophoresis to check the status and quality of the RNA.

### (2) Reverse Transcription Reaction

| | | |
|---|---|---|
| OligodT23 VN (50 uM) | 1 | μl |
| Total RNA | 1-2 | μg |

| | |
|---|---|
| RNase Free ddH2O | to 8 μl |

Above were heated at 65° C. for 5 minutes, quickly placed on ice to quench, and allowed to stand for 2 minutes.

Preparation of First-Strand cDNA Synthesis Solution

| | |
|---|---|
| 2 × RT Mix | 10 μl |
| HiScript II Enzyme Mix | 2 μl |

The first-strand cDNA synthesis was carried out according to the following conditions:

| | |
|---|---|
| 25° C. | 5 min |
| 50° C. | 45 min |
| 85° C. | 5 min |

### (3) Real-Time Quantitative PCR Reaction

The reverse transcription reaction product cDNA was diluted 50 times as a template.

| | | |
|---|---|---|
| AceQ SYBR Green Master Mix | 10 | μl |
| Primer 1 (10 uM) | 0.4 | μl |
| Primer 2 (10 uM) | 0.4 | μl |
| Rox Reference Dye | 0.4 | μl |
| Template | 2 | μl |
| ddH₂O to | 20 | μl |

Samples were loaded according to the above standard, and the reaction conditions were: 95° C. pre-denaturation for 15 seconds, then 95° C. for 5 seconds, 60° C. for 31 seconds, 40 cycles; melting curve conditions were 95° C. for 15 seconds, 60° C. for 30 seconds, 95° C. for 15 seconds. The sample was reacted on ABI ViiA7 (ABI) instrument. The expression of R-actin was used as an internal reference.

After the reaction was completed, the amplification of each gene was checked by software analysis, the corresponding threshold cycle number was derived, and the relative expression of each gene was calculated using the 2-ΔΔCt method. The peak and waveform of the melting curve was analyzed to determine whether the obtained amplification product was a specific single target fragment.

### 8. Western Blot Analysis

### (1) Extraction of Total Cellular Proteins

After simple washing with pre-chilled PBS buffer, cells were treated with RIPA cell lysis buffer (Invitrogen) containing 1 mM PMSF (protease inhibitor). The cells were lysed on ice for 30 minutes, and cell lysates were collected using a cell scraper. The collected lysates were centrifuged at 12,000 rpm for 15 minutes at 4° C., and the supernatants were carefully collected and stored at −80° C.

### (2) BCA Protein Quantification

The BCA protein quantification kit (Pierce) was used by mixing reagent A and reagent B in a ratio of 1:50 to prepare a working solution. Standard proteins were diluted to concentrations of 0 μg/μl, μg/μl, 50 μg/μl, 100 μg/μl, 250 μg/μl, 500 μg/μl, 750 μg/μl, 1000 μg/μl and 2000 μg/μl. In a microplate, 5 μl of standard protein or 5 μl of sample was added, followed by the addition of 100 μl BCA working solution. After thorough mixing, the plate was incubated at 37° C. for 30 minutes in a water bath, and the absorbance at 570 nm was read using a plate reader. Using the absorbance values as the y-axis and the concentrations of standard proteins as the x-axis, a standard curve was plotted.

Based on the standard curve, the concentration of the samples was calculated.

### (3) SDS-PAGE Electrophoresis

A 12% SDS-PAGE gel was prepared (a 5 ml system containing 2 ml of 30% acrylamide, 1.6 ml of ddH₂O, 1.3 ml of 1.5 μM pH8.8 Tris-HCl, 50 μl of 10% SDS, 50 μl of 10% ammonium persulfate and 2 μl of TEMED). After rapid mixing, the solution was poured into precast glass plates (Bio-Rad) and topped with an appropriate amount of deionized water to facilitate gel polymerization. The gel was left at room temperature for 30 minutes until complete polymerization, then the upper layer of deionized water was discarded, and any remaining liquid was dried with filter paper. A stacking gel was prepared (a 2 ml system containing 0.33 ml of 30% acrylamide, 0.25 ml of 1.0 μM pH6.8 Tris-HCl, 20 μl of 10% SDS, 20 μl of 10% ammonium persulfate and 2 μl of TEMED). This mixture was immediately added on top of the separating gel, with a clean 10-tooth comb inserted. The gel was left at room temperature for 30 minutes. After complete solidification of the stacking gel, the comb was removed. The gel was washed with ddH₂O several times in the well, and then placed in the electrophoresis chamber (Bio-Rad) with the addition of electrophoresis buffer (containing 25 mM pH8.0 Tris, 0.25 μM Glycine, 0.1% SDS). Protein samples were mixed with 6× loading buffer (containing 300 mM pH6.8 Tris-HCl, 12% SDS, 600 mM DTT, 60% glycerol, 0.6% bromophenol blue) at a ratio of 5:1, boiled for 10 minutes, cooled on ice for 5 minutes, and loaded into each lane according to the protein quantification results. Electrophoresis was carried out using a Bio-Rad electrophoresis apparatus, starting at 80V for approximately 20 minutes until the bromophenol blue front entered the separating gel. The voltage was then increased to 120V, and electrophoresis was continued for about 1 hour until the bromophenol blue band reached the bottom of the separating gel. The electrophoresis was completed.

### (4) Protein Transfer

After SDS-PAGE electrophoresis, areas in the stacking gel without samples were removed, and the nitrocellulose membrane was briefly soaked in transfer buffer. In the transfer device (Bio-Rad), from anode to cathode, Bio-Rad 3 mm filter paper, nitrocellulose membrane, gel, and Bio-Rad 3 mm filter paper were placed in sequence. Transfer was performed at 100V for 1.5 hours. After the transfer, the membrane was stained with Marker and 0.1% Ponceau Stain in advance for 5 minutes to determine the transfer effect, followed by decolorization with ddH2O.

### (5) Antibody Labeling and ECL Detection

The nitrocellulose membrane was blocked in a blocking solution (TBST containing 5% non-fat milk) at room temperature for 1 hour. It was then incubated overnight in primary antibody hybridization solution at 4° C. The membrane was washed three times with TBST at room temperature, each time for 2 minutes. HRP-conjugated secondary antibody hybridization solution, prepared in blocking solution, was added at room temperature and incubated for 0.5 hours. The membrane was washed three times with PBST at room temperature, each time for 2 minutes.

Equally proportional substrate and enhancer in SuperSignal West Pico kit (Pierce) were mixed. The mixture was evenly dropped onto the membrane, incubated at room temperature for 1 minute. X-ray film exposure was performed. After developing and fixing, the X-ray film was scanned and saved for analysis.

### 9. SA-β-Gal Staining

Senescence-associated β-galactosidase (SA-β-Gal) staining was performed following a previously reported procedure (Debacq-Chainiaux et al., 2009). Briefly, cells in culture dishes were washed with PBS and fixed at room temperature. Cells were fixed in 2% formaldehyde and 0.2% glutaraldehyde for 3 minutes. SA-β-Gal was used for staining with freshly prepared staining solution overnight at 37° C. Images were taken on the next day and the percentage of positive cells per unit area was calculated.

10. Tissues Obtained from Clinical Patients with Prostate Cancer and Breast Cancer and Biospecimen Analysis

In the present disclosure, administration of chemotherapeutic agents was performed for Chinese patients with prostate cancer (Clinical Trials no. NCT03258320) and patients with infiltrating ductal breast cancer (NCT02897700) by the pathological characteristics. Patients with a clinical stage ≥I subtype A (IA) (TIa, NO, MO) of primary prostate cancer but without manifest distant metastasis were enrolled into the clinical cohorts. At the same time, age >18 years with histologically proven infiltrating ductal BCa was required for recruitment. All patients were provided with an informed consent form and signed to confirm their agreement. Data regarding tumor size, histological type, tumor penetration, lymph node metastasis and pathological TNM stage were obtained from the pathological records. Tumors were processed as formalin-fixed paraffin-embedded biospecimens and sectioned for histological assessment, with selectively isolated OCT-frozen slides processed via LCM for gene expression analysis. Specifically, stromal cells associated with glands and adjacent to the cancer epithelium were separately isolated from tumor biopsies before and after chemotherapy using LCM following previously defined criteria (Sun et al., 2012). Immunoreactive scores (IRS) were categorized according to staining intensity per tissue sample into four groups including 0-1 (negative), 1-2 (weak), 2-3 (moderate) and 3-4 (strong) (Fedchenko and Reifenrath, 2014). The diagnosis of PCa and BCa in tissues was confirmed based on histological evaluation by independent pathologists. Randomized controlled trial (RCT) protocols and all experimental procedures were approved by the IRB of the Shanghai Jiao Tong University School of Medicine, with methods carried out in accordance with the official guidelines.

### 11. Tumor Xenografting in Mice and Pre-Clinical Chemotherapeutic Procedures

All experimental mouse experiments were carried out in strict accordance with the relevant regulations of the Institutional Animal Care and Use Committee (IACUC), Shanghai Institutes for Biological Sciences, Chinese Academy of Sciences. Immuno-deficient mice ICR SCID mice (body weight about 25 g) aged about 6 weeks were used for animal experiments related to the present disclosure. Stromal cells PSC27 and epithelial cells were mixed at a ratio of 1:4, and each graft contained 1.25×10⁶ cells for tissue remodeling. The xenograft tumors were implanted into mice by subcutaneous transplantation, and the animals were euthanized 8 weeks after the end of the transplantation surgery. Tumor volume was calculated according to the following formula: V=(π/6)×((1+w)/2)³ (V, volume; 1, length; w, width).

For pre-clinical chemotherapeutic studies, mice received subcutaneous implantation were given standard laboratory diets. After 2 weeks, a chemotherapeutic drug mitoxantrone (MIT, 0.2 mg/kg doses), doxorubicin (DOX, 1.0 mg/kg doses), paclitaxel (PTX, 1.0 mg/kg doses) or docetaxel (DTX, 1.0 mg/kg doses), and/or a PDK4 inhibitor (PDK4-IN, 500 l, 10.0 mg/kg doses) was administered by intraperitoneal injection. The time points were on the first day of the third, fifth and seventh weeks, with a total of 3 cycles of treatment, and each cycle lasted for 2 weeks. After the course of treatment, mouse kidneys were collected for tumor measurement and histological analysis. Each mouse received a cumulative dose of 0.6 mg/kg body weight of mitoxantrone (3.0 mg/kg body weight of doxorubicin, 3.0 mg/kg body weight of paclitaxel, 3.0 mg/kg body weight of docetaxel, 30 mg/kg body weight of PDK4 inhibitor). In order to cause systemic expression of SASP factors induced by chemotherapy, MIT was administered to mice through intravenous infusion according to the above steps and sequence, but the dose was reduced to 0.1 mg/kg body weight/each time (the cumulative dose of MIT received in the whole course of treatment was 0.3 mg/kg body weight) to reduce drug-related toxicity. The chemotherapy experiment ended at the end of the 8th week, and the mice were dissected immediately after sacrifice, and the xenograft tumors were collected and used for pathological system analysis.

### 12. Study on Mouse Lifespan

For the cell transplantation study, the inventors obtained 16-month-old male C57BL/6 mice by serial rearing in the SPF animal platform, with 4 to 5 animals per cage. The inventors first sorted the mice by body weight from low to high, and then selected mice with similar body weights. Next, the mice were assigned to one treatment group at each interval using a random number generator, while the middle mice were assigned to the other treatment group, so that the body weights of mice in the senescent group and drug control group were matched. One month after cell transplantation, when the mice were 18 months old, physical function tests were performed. After that, no further testing was done on the mice other than checking their cages.

The earliest deaths occurred approximately 2 months after the last physical function test. C57BL/6 mice aged 19 to 21 months were housed at 3-5 animals per cage. Same as the transplanted mice, the mice were sorted by body weight and randomly assigned to each group, and treated in either control (vehicle) or drug (PDK4) group by persons blinded to the design of the preclinical trial. From the age of 24-27 months, the mice were treated with vehicle or PDK4-IN every 2 weeks, orally gavaged for 3 consecutive days at each time. During the course of the study, some mice were removed from their original cages in order to minimize the animal housing stress caused by long-term housing in a single cage. RotaRod and hanging tests were performed monthly because these tests were sensitive and noninvasive. At the end of the experiment, the mice were euthanized and considered to be dead if they exhibited one of the following symptoms: (i) unable to drink or eat; (ii) unwilling to move even when stimulated; (iii) rapid weight loss; (iv) severe balance disorder; or (v) body bleeding or ulcerated tumors. During the experiment, no mice were excluded due to fighting, accidental death or dermatitis. For biostatistics, a Cox proportional hazard model was used for survival analysis.

### 13. Physical Function Tests

All testing began on day 5 after the last placebo or drug treatment. Maximum walking speed was assessed using the accelerated RotaRod System (TSE System, Chesterfiled, MO). The mice were trained on the RotaRod for 3 days at speeds of 4, 6 and 8 r.p.m, lasted for 200 seconds on the 1st, 2nd and 3rd days. On the test day, the mice were placed on the RotaRod, and the test was performed starting at a speed of 4 r.p.m. The rotation speed was accelerated from 4 to 40 r.p.m at intervals of 5 minutes. When the mice fell off the RotaRod, the speed was recorded. The final results were averaged from 3 or 4 trials and normalized to baseline speed. The mice that had been trained within the previous two months were no longer trained.

Forelimb grip strength (N) was measured using a Grip Strength Meter (Columbus Instruments, Columbus, OH), and the results were averaged from more than 10 trials. For the hanging endurance test, the mice were placed on a 2 mm thick metal wire, while the later was 35 cm above a mat. The mice were only allowed to grasp the wire with their forelimbs, and the hang time was normalized to body weight and expressed as hang duration (sec)×body weight (g). The results were the average of 2 to 3 trials per mouse. Their daily activity and food intake were monitored for 24 h (12 hours of light and 12 hours of dark) by a Comprehensive Laboratory Animal Monitoring System (CLAMS). The CLAMS system was equipped with an Oxymax Open Circuit Calorimeter System (Columbus Instruments). For treadmill performance, the mice were acclimatized to run on an electric running machine (Columbus Instruments) at a 5° incline, for 3 days of training, lasted for 5 minutes each day, started at a speed of 5 m/min for 2 minutes, then accelerated to 7 m/min for 2 minutes, then 9 m/min for 1 minute. On the day of the test, the mice ran on the treadmill at an initial speed of 5 m/min for 2 min, and then the speed increased by 2 m/min every 2 min until the mice were exhausted. Fatigue was defined as the inability of mice to return to the treadmill despite mild electrical and mechanical stimulation. The distance was recorded after the test, and the total work (KJ) was calculated with the following formula: mass (kg)×g (9.8 m/s2)×distance (m)×sin (5°).

### 14. Post-Mortem Pathological Examination of Preclinical Animals

The researchers checked the cages daily and removed dead mice from the cages. Within 24 hours of animal death, cadavers (abdominal cavity, thoracic cavity and skull) were opened and kept separately in 10% formalin for at least 7 days. Decomposed or destroyed bodies were excluded. The preserved cadavers were transported to the dedicated autopsy site for pathological examination. Tumor burden (sum of different types of tumors per mouse), disease burden (sum of different histopathological changes in major organs per mouse), severity of each lesion and inflammation (lymphocyte infiltration) were assessed.

### 15. Biostatistical Methods

In the present patent application, all in vitro experiments involving cell proliferation rate, survival rate and SA-β-Gal staining, and in vivo experiments on mouse xenograft tumors and preclinical drug treatment were repeated more than 3 times, and the data were presented in the form of mean±standard error. The statistical analyzes were established on the basis of raw data and calculated by one-way analysis of variance (ANOVA) or a two-tailed Student's t-test, while the results with P<0.05 were considered to be significantly different.

The correlation between factors was tested by Pearson's correlation coefficients. Cox proportional hazards model was used for survival analysis when mice were obtained in several cohorts and grouped in cages. In the model, sex and age were used in the treatment as fixed effects, while cohort and initial cage assignment were used as random effects. Since during the study some mice were moved from their initial cages to minimize stress from the single cage enclosure, we also performed analysis without cage effects. The results of the two analyzes did not differ significantly in directionality or statistical significance, which increased confidence in results. Survival analysis was performed using the statistical software R (version 3.4.1; library “coxme”). In most experiments and outcome assessments, investigators made blind selections for assignments. The inventors used baseline body weights to assign mice to experimental groups (to achieve similar body weights between groups), so randomization was only performed within groups matched for body weight. The sample size was determined based on previous experiments and therefore did not use statistical power analysis. All replicates in this study were derived from different samples, and each sample was derived from a different experimental animal.

### Second Part: Examples

### Example 1. DNA Damaging Induces Cellular Senescence, Accompanied by High Expression of PDK4

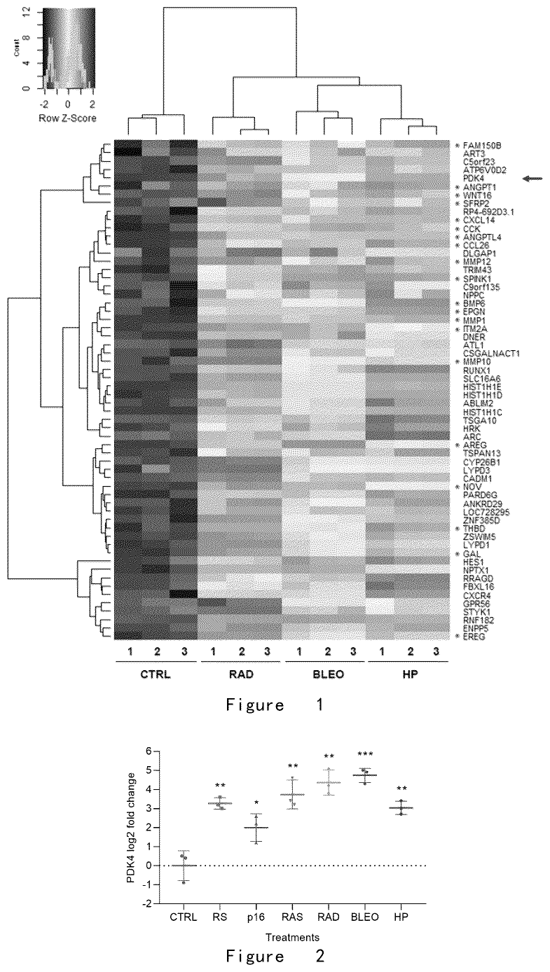

After extensive analysis and studies, the inventors noticed a stromal cell line, namely PSC27, originating from human prostate tissue. This cell line mainly comprises fibroblasts but also comprises a small fraction of non-fibroblast lines, such as endothelial and smooth muscle cells. When exposed to cytotoxic damage, these cells have the capability to produce a significant amount of SASP factors. Interestingly, PDK4 as one of the key factors, together with a list of typical SASP components, upregulated significantly, as revealed by previous microarray profiling (FIG. 1). To confirm this finding, the inventor used several alternative approaches to induce senescence, including replicative exhaustion (RS), p16^{INK4a} (p16) and overexpression of HRAS^{G12V} (RAS). These stress inductions led to comprehensive cellular senescence, with efficacy similar to DNA damages such as radiation (RAD), bleomycin (BLEO), and hydrogen peroxide (HP) (SA-β-Gal positivity and BrdU incorporation). In each case, the inventors observed that there was an obviously induced expression of PDK4 (FIG. 2-3).

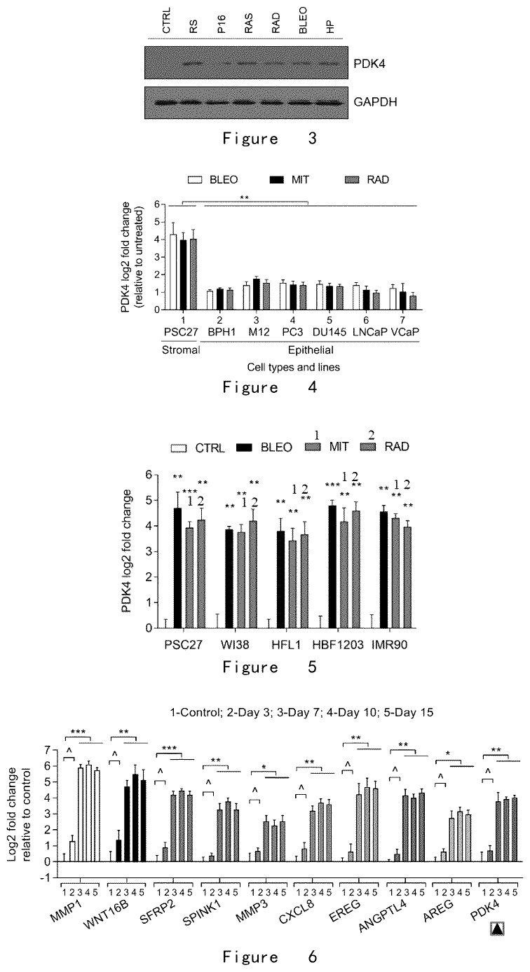

Expression analysis of several cell lines of human prostate origin suggested that stromal cells are indeed more PDK4-inducible than cancer epithelial cells, implying a special mechanism supporting PDK4 production in prostate stromal cells (FIG. 4). Data from several additional fibroblast lines consistently supported a robust induced expression of PDK4 upon genotoxic treatment by antitumor drugs (FIG. 5). Notably, the transcriptional expression pattern of PDK4 resembled that of a series of typical SASP factors, including MMP1, WNT16B, SFRP2, SPINK1, MMP3, CXCL8, EREG, ANGPTL4 and AREG, which exhibited a gradual increment until cells entered a platform within 7-8 days after treatment (FIG. 6-7). In the human PDK family (PDK1-PDK4 isozymes), PDK4 seemed to be the only member easily inducible by genotoxic stress, with a tendency similar to that of CXCL8, an index of SASP expression (FIG. 8).

### Example 2. PDK4 Expression in Stroma Predicts Adverse Clinical Outcomes after Chemotherapy

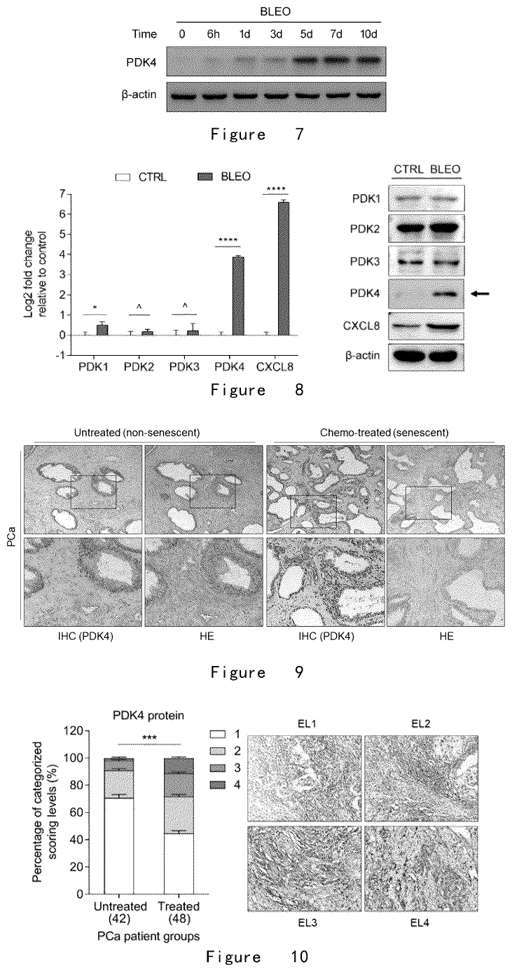

The in vitro results prompted the inventors to further determine whether PDK4 induction occurs within the tumor microenvironment (TME), a pathological entity where many benign stromal cells reside. The inventors first chose to analyze clinical samples of a cohort of patients with prostate cancer (PCa) who developed primary tumors in prostate and underwent neoadjuvant regimen involving a genotoxic agent (mitoxantrone; MIT). Surprisingly, PDK4 was found markedly expressed in prostate tissues of these patients after neoadjuvant chemotherapy, but not before (FIG. 9). In line with in vitro data of the inventors, upregulated PDK4 was generally localized in stroma, in a sharp contrast to the adjacent cancer epithelium, which had limited or no staining.

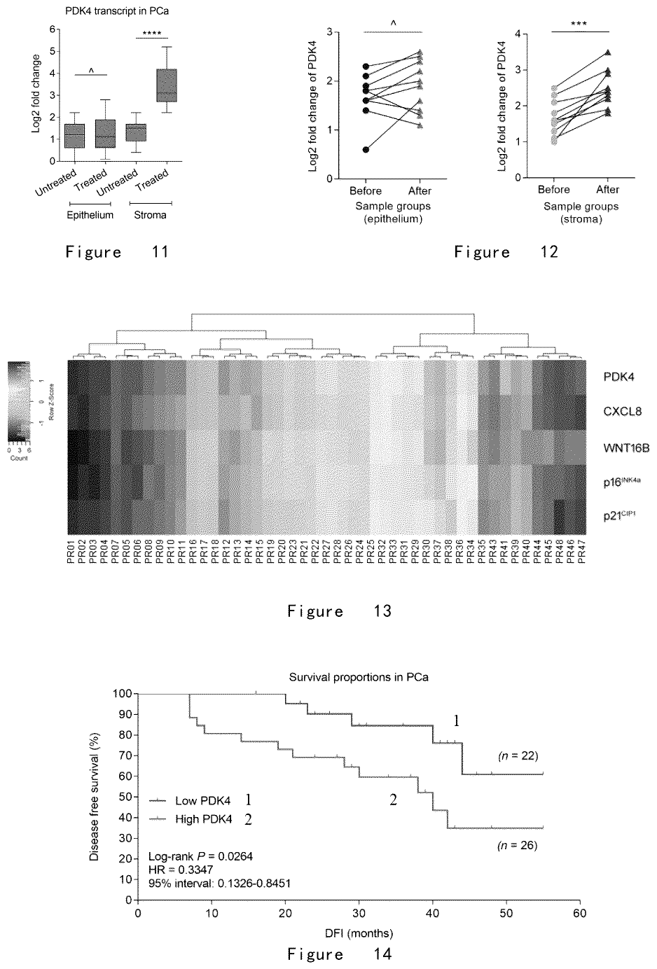

By a pre-established pathological assessment procedure, PDK4 production in patient samples pre-versus post-chemotherapy was quantitatively measured. This allowed precise evaluation of a target protein per immunohistochemistry (IHC) staining intensity to be possible (FIG. 10). Transcript analysis upon laser capture microdissection (LCM) of cell lineages from primary tissues suggested that PDK4 was more readily induced in stroma rather than cancer compartments (P<0.0001 vs P>0.05) (FIG. 11). To substantiate PDK4 inducibility in vivo, the inventors profiled a subset of patients with PCa whose pre- and post-chemotherapeutic biospecimens were both accessible, and found notably upregulated PDK4 in stroma, but not cancer epithelium, of each individual post-chemotherapy (FIG. 12). The inventors noticed that the dynamics of PDK4 expression in the damaged TME were largely in parallel with that of CXCL8 and WNT16B (FIG. 13). The expression pattern of these factors was largely consistent with that of senescence markers p16^{INK4a} and p21^{CIP1} in tumor foci, suggesting an inherent correlation of PDK4 induction with cellular senescence (FIG. 13). Of note, Kaplan-Meier analysis of patients with PCa stratified according to PDK4 expression in the tumor stroma suggested a significant but negative correlation between PDK4 protein level and disease-free survival (DFS) in the treated cohort (P<0.05, Log-rank test) (FIG. 14).

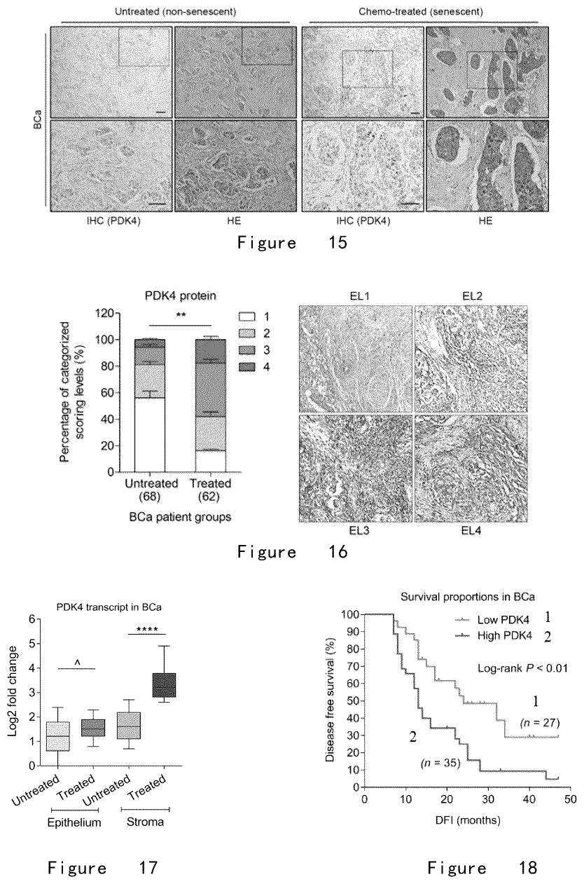

The distinct pathological properties of PDK4 in patients with tumor were subsequently reproduced by an extended study involving clinical cohorts of human patients with breast cancer (BCa) (FIGS. 15-18).

### Example 3. Senescent Cells Exhibit a Distinct Glucose Metabolism Profile

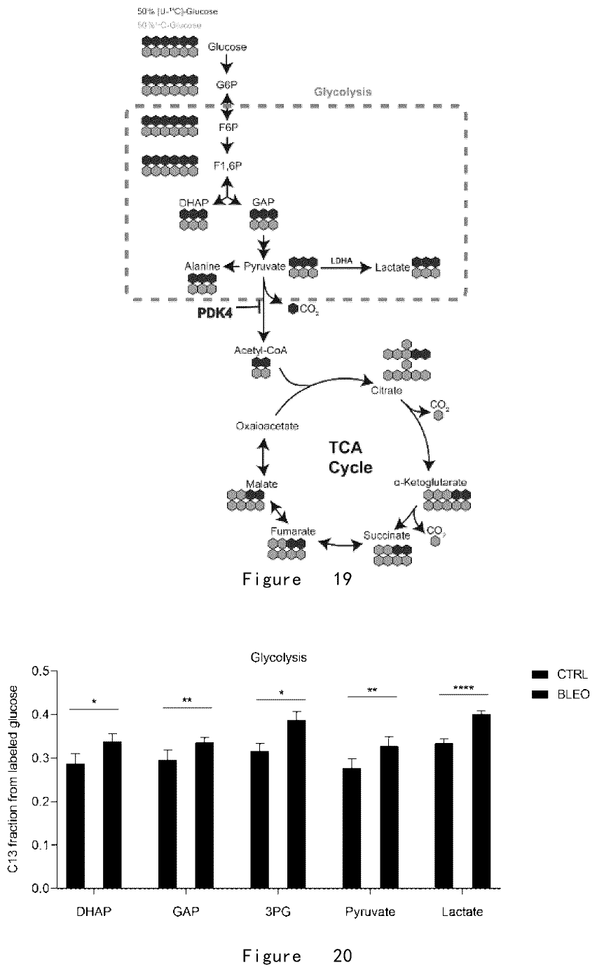

Glucose is the primary carbon source to the tricarboxylic acid (TCA) cycle, followed by glutamate and aspartate (non-protonatable amino acids as glutamine or asparagine, respectively) as secondary sources (FIG. 20). The inventors first interrogated the metabolic pattern of glucose upon uptake by senescent cells, as glucose acts as a principal contributor to TCA cycle when cells enter senescence, a stage allowing cells to sustain metabolic activity. Experimental data from analysis of mitochondrial dynamics and cellular bioenergetics with gas chromatography-mass spectrometry (GC-MS) indicated notably elevated glycolytic activity in senescent human stromal cells, as reflected by enhanced production of metabolites, including but not limited to dihydroxyacetone phosphate (DHAP), glyceraldehyde-3-phosphate (GAP) and 3-phosphoglycerate (3-PG) (FIG. 20). Increased levels of GAP and 3-PG imply further utilization of a number of middle metabolites, such as citrate, α-ketoglutarate, glutamate, succinate, succinate, fumarate and malate, all metabolically derived from pyruvate and substantiated by metabolic profiling with GC-MS (FIG. 21). In summary, bioactivities of both glycolysis and the TCA cycle were significantly enhanced in senescent cells, as reflected by metabolic profiling with assays of stable isotope labeling with a uniformly labeled U-13C6 glucose tracer and fractioning of metabolites derived from labeled glucose and revealed by GC-MS (FIG. 22). Notably, entry of glucose-derived and PDH-catalyzed flow of carbon into the TCA cycle generates isotopomer species with two labeled carbons (M2), whereas species with more labeled carbons (M3 and M4) arise from the addition of labeled acetyl-CoA to labeled oxaloacetate produced by TCA cycling (FIG. 19). Compared to proliferating cells, senescent cells displayed an increased rather than decreased citrate M2/pyruvate M3 ratio, further implying enhanced TCA cycle activity alongside the simultaneously increased glycolytic capacity (FIG. 23), a feature that makes them remarkably distinct from various cancer cell types.

The inventors noticed that these metabolic changes were accompanied by substantial perturbations in mitochondrial ultrastructure of senescent cells, particularly enlarged sizes and abnormal shapes as revealed by transmission electron microscopy (TEM). These tendencies indicated ultrastructural damage of mitochondria, suggesting potential mitochondrial dysfunction associated with oxidative stress upon cellular senescence (FIG. 24). These observations are largely in line with former studies regarding abnormal phenotypes of mitochondria including mass, dynamics and structure upon senescent cells.

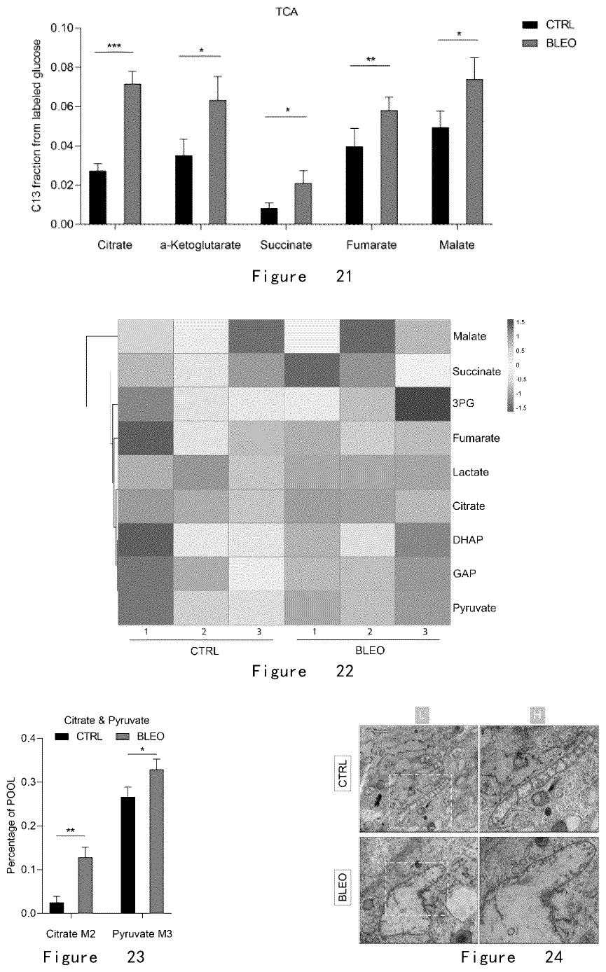

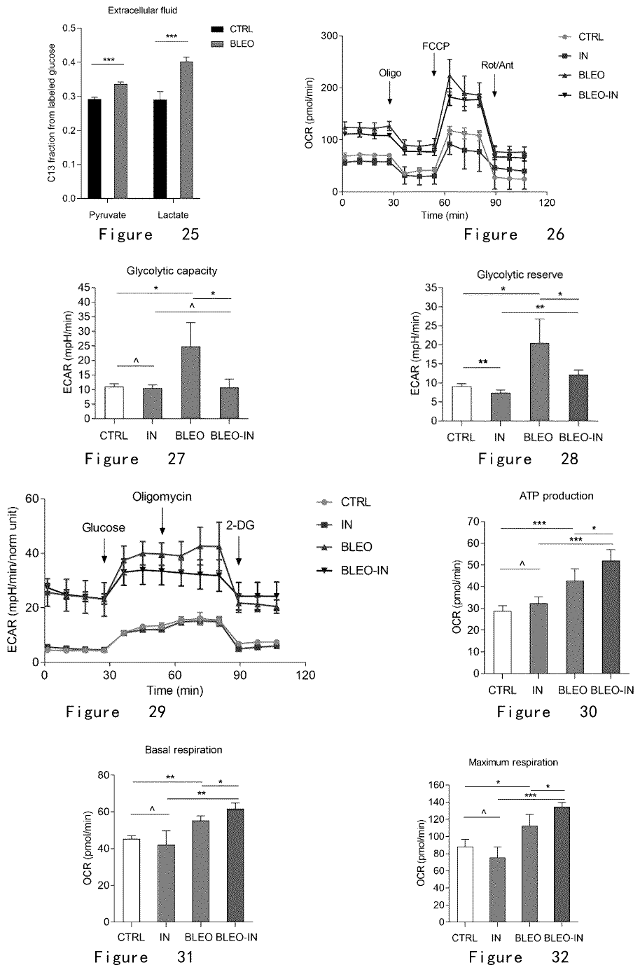

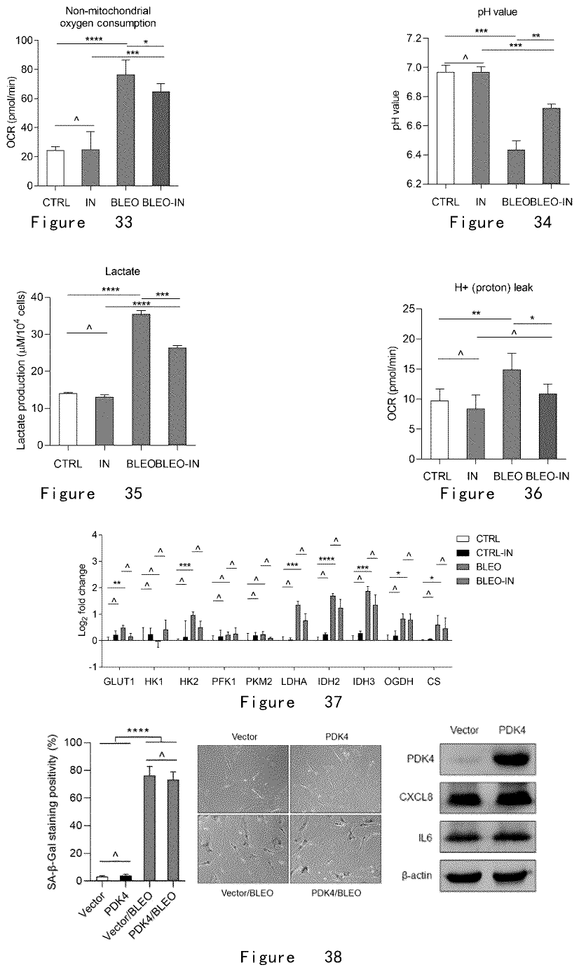

Next, the inventors measured the levels of extracellular fluids. Notably, amounts of both pyruvate and lactate released to the extracellular space were significantly enhanced in senescent cells (FIG. 25). As determined by an XF24 Extracellular Flux Analyzer (Seahorse Bioanalyzer), these changes were accompanied by alterations in oxygen consumption rate (OCR) and extracellular acidification rate (ECAR), suggesting elevated metabolic activities associated with glucose utilization (FIGS. 26-29). Correspondingly, the inventors observed elevated ATP production, basal respiration, maximal respiration in senescent cells, a pattern indicative of tight connection of the TCA cycle and oxidative phosphorylation (OXPHOS) but further promoted when PDK4-IN-1 (CAS No. 2310262-10-1, an anthraquinone derivative and a potent inhibitor of PDK4, abbreviated as PDK-IN hereafter, with a final concentration of 5 M) was applied to culture (FIGS. 30-32). However, treatment with PDK4-IN reversed changes in non-mitochondrial oxygen consumption, pH fluctuation, lactate production and H⁺ (proton) leak, with the overall metabolic data validated by principal-component analysis (PCA) (FIGS. 33-36). The inventors next found that these alterations occurred in parallel with expression changes of glucose uptake-associated molecules and metabolism-related enzymes, including glucose transporter 1 (GLUT1), hexokinase 2 (HK2), lactate dehydrogenase A (LDHA), isocitrate dehydrogenase 2 (IDH2), isocitrate dehydrogenase 3 (IDH3), oxoglutarate dehydrogenase (OGDH) and citrate synthase (CS) (FIG. 37). Among them, HK2 and LDHA are glycolysis-related factors, whereas IDH2, IDH3, OGDH and CS are TCA cycle-involving enzymes. As overexpression of PDK4 per se in normal cells neither caused or abrogated senescence, nor affected the SASP (FIG. 38), the inventors reasoned that senescent cell metabolism was correlated with and likely underpinned by expression of key factors involved in glucose consumption and linked with production of pyruvate, lactate and multiple other metabolites. Notably, elevated levels of glycolysis and oxidative phosphorylation were simultaneously observed (FIGS. 27, 31, 32), suggesting essentially reprogrammed glucose metabolism upon senescence.

Experimental data confirmed that compared with proliferative cells, senescent cells exhibit increased glucose transporter and glycolytic enzyme expression after chemotherapeutic treatment (FIG. 37). Compared with the control group, steady-state glucose concentrations tend to be higher in senescent cells, suggesting an elevated glucose avidity upon senescence. These findings are also substantially confirmed by metabolomics profiling, which underscores the global catabolic nature of senescence-associated metabolic changes. However, PDK4 per se neither influenced cell senescence, nor affected the expression level of typical SASP factors (FIG. 38).

Together, senescent cells develop a distinctive hypermetabolic phenotype characterized of enhanced glycolysis, TCA cycle activity and ATP-boosting oxidative phosphorylation. Increased energy production is a common denominator of senescent cells, which exhibit specific utilization of energy-generating metabolic pathways, a phenomenon partially reminiscent of the ‘Warburg effect’ observed in cancer cells capable of non-oxidative glycolysis.

### Example 4. Senescent Cells Produce Lactate Via Glucose Consumption by PDK4 High Expression

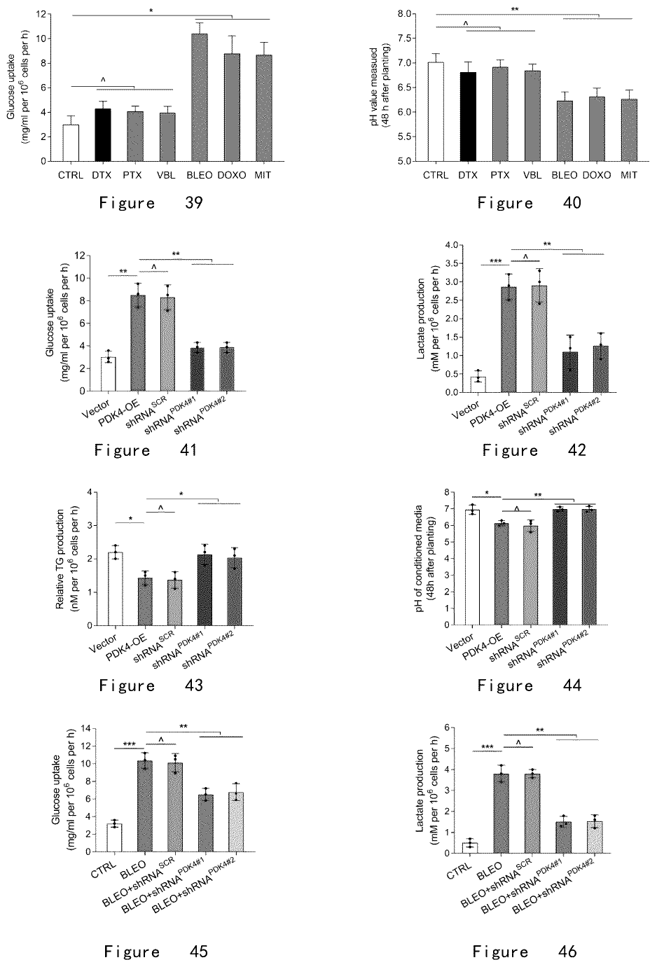

Senescent cells are in a hypermetabolic status, more specifically, these cells display a hypercatabolic nature, thus prompting the inventors to interrogate whether these cells have a glucose uptake capacity distinct from proliferating cells. To accurately answer this question, the inventors performed another set of metabolic assays. The results showed a significant increase of glucose uptake by senescent cells, although changes were preferentially detected upon genotoxicity-induced senescence (GIS), which usually involves DNA damaging agents (FIG. 39). In addition, the pH of conditioned medium (CM) from senescent cells was markedly decreased, a property that again seemed to be more dramatic for genotoxicity-induced senescence (FIG. 40). Given the results indicative of elevated acidification as revealed by ECAR assay (FIG. 29), the inventors reasonably speculated extracellular formation of an acidic microenvironment by senescent cells, whose metabolism seemed to be markedly reprogrammed and characterized with increased secretion of acidic metabolites. Data of the inventors indicated that senescent cells generated an increased amount of lactate, in contrast to their cycling controls (FIG. 39). A series of studies reported that cancer cells exhibit increased lactate production, OCR level and ATP output, a series of metabolic changes correlated with enhanced glycolysis.

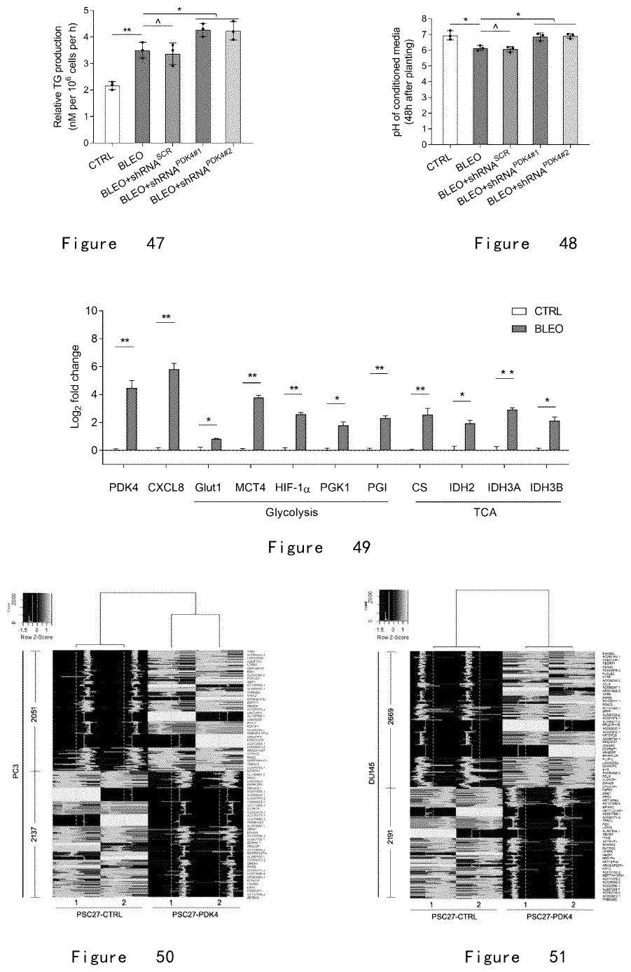

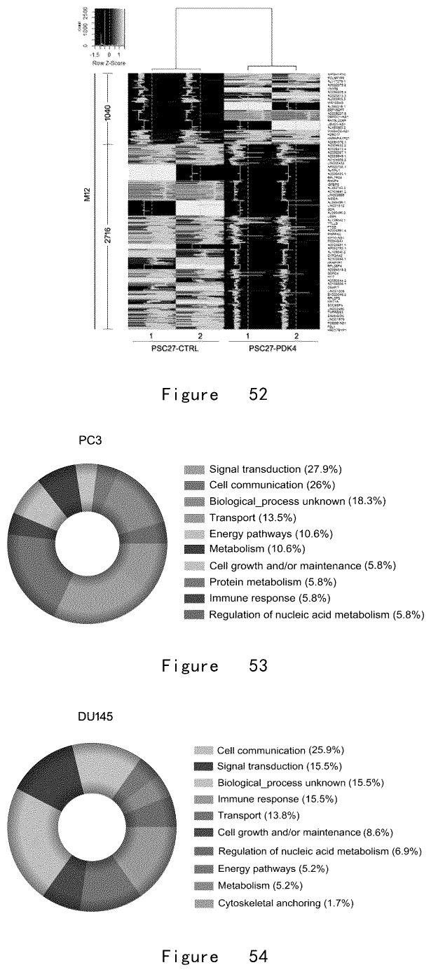

PDK4 is a key enzyme involved in regulation of glucose and fatty acid metabolism as well as tissue homeostasis, while its overexpression inactivates the PDH complex by phosphorylating the targets and contributes to metabolic flexibility. The inventors assessed the influence of PDK4 expression by transducing a PDK4 gene ORF to human stromal cells and noticed significantly altered metabolic profile, including glucose uptake, lactate and triglyceride (TG) production, although these changes were largely reversed upon genetic eliminated of PDK4 (FIGS. 40-42). It is worth noting that pH of the CM decreased upon PDK4 overexpression in proliferating cells, but subject to counteraction by PDK4 suppression (FIG. 44). The inventors further measured these parameters with BLEO-induced senescent cells and found markedly increased glucose uptake, lactate and TG production, but reduced pH of the CM (FIGS. 45-48). However, almost all these metabolic changes were substantially reversed upon PDK4 depletion in PSC27. This suggested that PDK4 mediated antagonism against TG synthesis throughout the TCA cycle in senescent cells (FIGS. 44-48). The inventors noticed that factors functionally supporting glycolysis and TCA, including GLUT1, MCT4, HIF1a, PGK1, PGI, CS, IDH2, IDH3A and IDH3B were concurrently upregulated upon GIS, further indicating an overall enhancement of cellular metabolism with glucose as the source of energy (FIG. 49).

### Example 5. PDK4 Positive Senescent Cells Alter the Expression Profile and Malignant Phenotype of Cancer Cells Through HTR2B

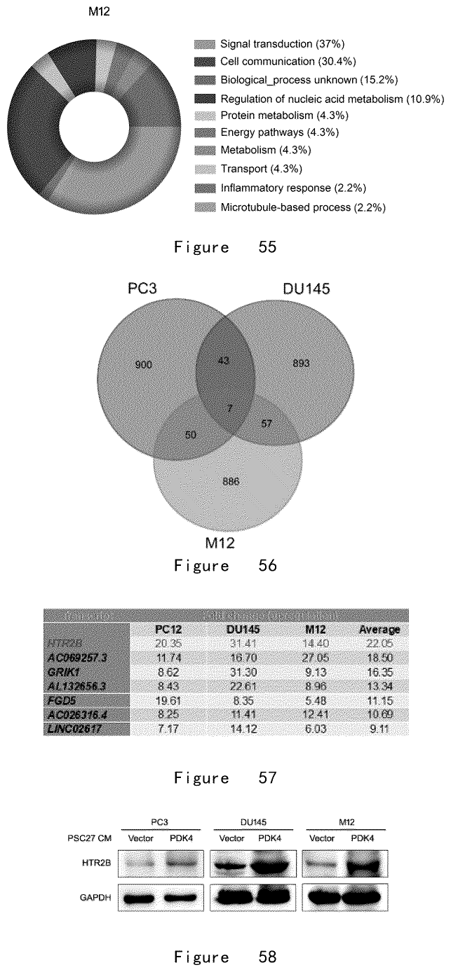

Next, the inventors sought to determine the influence of PDK4-expressing stromal cells on their surrounding microenvironment, especially cancer cells. As PSC27 is originally derived from the human prostate, the inventors first chose to examine PCa cells. PSC27-derived CM was prepared to treat PCa cells in culture, with cancer cells subject to genome-wide analysis. Data from RNA sequencing (RNA-seq) indicated 4,188 transcripts significantly upregulated or downregulated (FC>2, P<0.05) in PC3 cells, with 4,860 and 3,756 transcripts changed in DU145 and M12 cells, respectively (FIGS. 50-52). The inventors noticed remarkable and comprehensive changes in the biological processes of PCa cells, as evidenced by considerably affected activities in signal transduction, cell communication, intracellular transport, energy pathways and metabolism regulation (FIGS. 53-55). Overall, these data suggested PDK4-expressing stromal cells reprogrammed transcriptomic expression of recipient cancer cells by influencing CM, while CM showed a typical salient capacity.

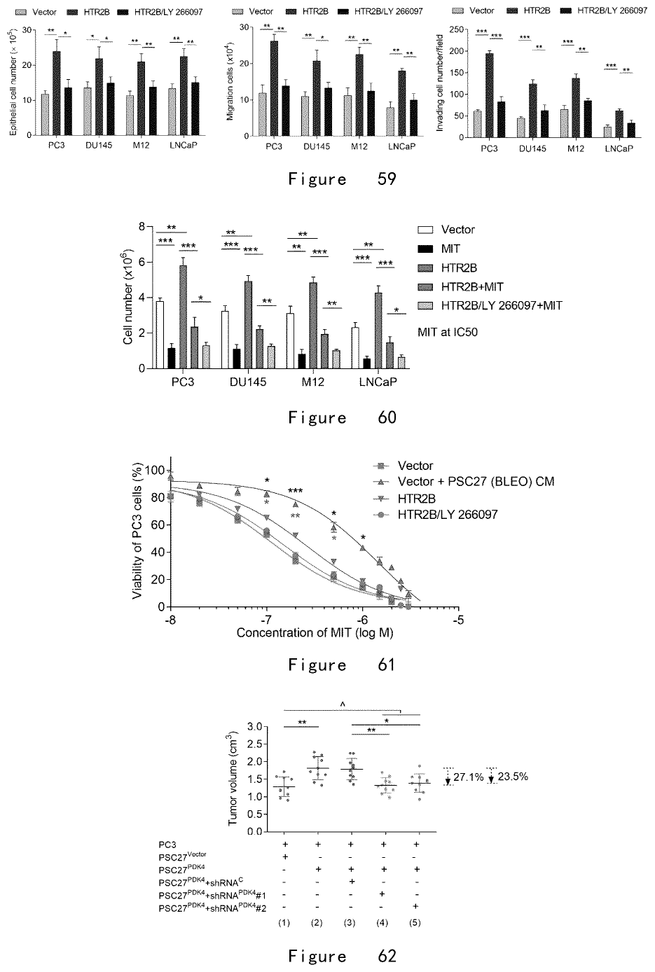

Among the transcripts significantly upregulated by PSC27 cell-derived CM (P<0.05, FDR<0.01, top 1,000), the inventors noticed that there were seven transcripts showing up and commonly expressed by PC3, DU145 and M12 cells (FC>4, P<0.01) (FIGS. 56-57). Next, the inventors focused on a specific gene (transcript) and determined whether it could potentially alter the malignancy of cancer cells. Immunoblotting confirmed its expression as 5-hydroxytryptamine receptor 2B (HTR2B), encoding several different serotonin receptors belonging to the G protein-coupled receptor 1 family and playing a crucial role. HTR2B was significantly expressed in PCa cells induced by the expression of PDK4 in stromal cell-conditioned medium (FIG. 58). Serotonin is a biological hormone, acting as a neurotransmitter and mitogen. Serotonin receptors mediate central and peripheral physiological functions, including regulation of cardiovascular functions and impulsive behaviors. The inventors explored the potential significance of HTR2B in the phenotypic changes of individual PCa cell lines.

TR2B transduction significantly enhanced capacity of proliferation, migration and invasion of PCa cells (FIG. 59). More importantly, the transduction of HTR2B enhanced resistance of PCa cells to mitoxantrone (MIT). MIT is a DNA-targeting chemotherapeutic agent administered to patients with cancer, including those developing PCa (FIG. 60). Survival curves of cancer cells under genotoxic stress of MIT displayed a declined trend depended on concentrations of this drug, as exemplified by the case of PC3 (FIG. 61). Notably, the CM (PSC27(BLEO)CM) produced by senescent stromal cells after BLEO treatment exhibits a higher increase in HTR2B expression, leading to a more significant enhancement of chemotherapy resistance. This suggested that the presence of other molecules with specific roles in the CM of senescent stromal cells, particularly a large amount of soluble proteins within the broad spectrum of the SASP. However, the acquisition of these functions was generally lost when the selective antagonist of HTR2B, LY 266097 (CAS No. 172895-39-5), was applied under culture conditions. This suggested that the malignant enhancement of cancer cells is mainly attributed to the activity of ectopically expressed HTR2B in these cell lines (FIGS. 59-61).

### Example 6. Therapeutically Targeting PDK4 Improves Chemotherapeutic Efficacy in Preclinical Trials

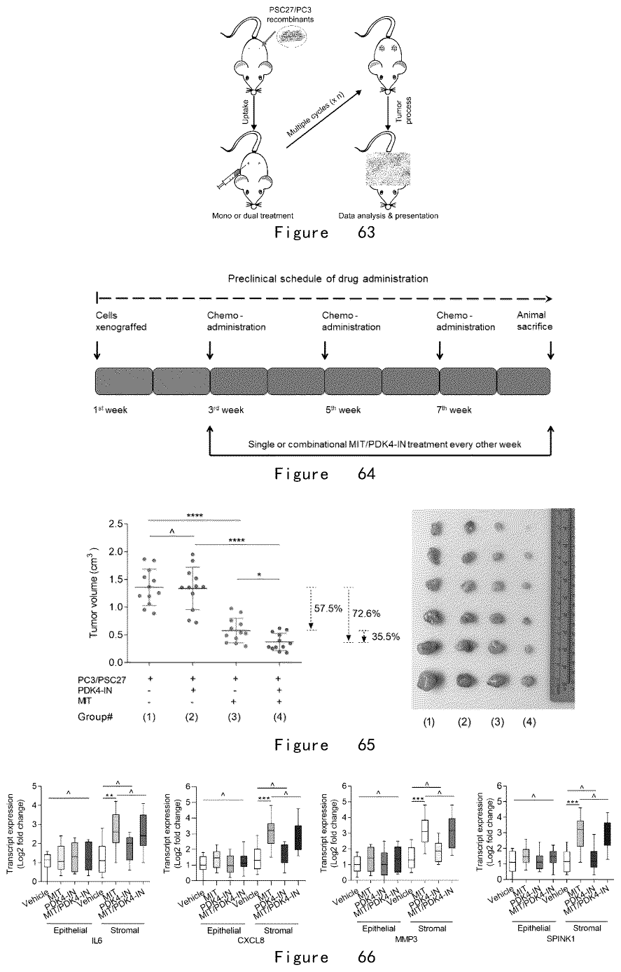

Given that acidic microenvironment formed by PDK4 highly-expressing stromal cells and its effects on cancer cell expression and phenotypes in vitro, the inventors queried the pathological consequences in TME of PDK4 induction in vivo. The inventors chose to carry out subcutaneous cell implantation to hind flank of experimental mice with severe combined immunodeficiency (SCID). Tissue recombinants were constructed by admixing PSC27 sublines with PC3 cells at a pre-optimized ratio. Animals were gauged for tumor size at end of an 8-week period. Compared to tumors consisting of PC3 and PSC27 vector (blank expression vector), xenografts consisting of PC3 and PSC27PDK4 (overexpressed PDK4) displayed significantly increased sizes (P<0.01) (FIG. 62). Conversely, PDK4 knockdown (by shRNA) from these PSC27PDK4 cells before xenograft implantation markedly reduced tumor volumes (P<0.01 and P<0.05).

To closely mimic clinical conditions involving chemotherapeutic drugs, the inventors designed a preclinical regimen incorporating a genotoxic drug (MIT) and/or the PDK4 inhibitor (PDK4-IN) (FIG. 63). Two weeks after cell implantation when stable uptake of tumors by host animals was generally observed, a single dose of MIT or placebo was administered at the first day of the third, fifth and seventh week until end of the 8-week regimen (FIG. 64). Although PDK4-IN administration did not provide noticeable benefits, MIT treatment caused notable tumor shrinkage (57.5% reduction), validating the efficacy of MIT as a cytotoxic drug (FIG. 65). Importantly, when PDK4-IN was combined with MIT, a further decline of tumor volume was observed (35.5% reduction), resulting in a total shrinkage by 72.6% compared to the control group.

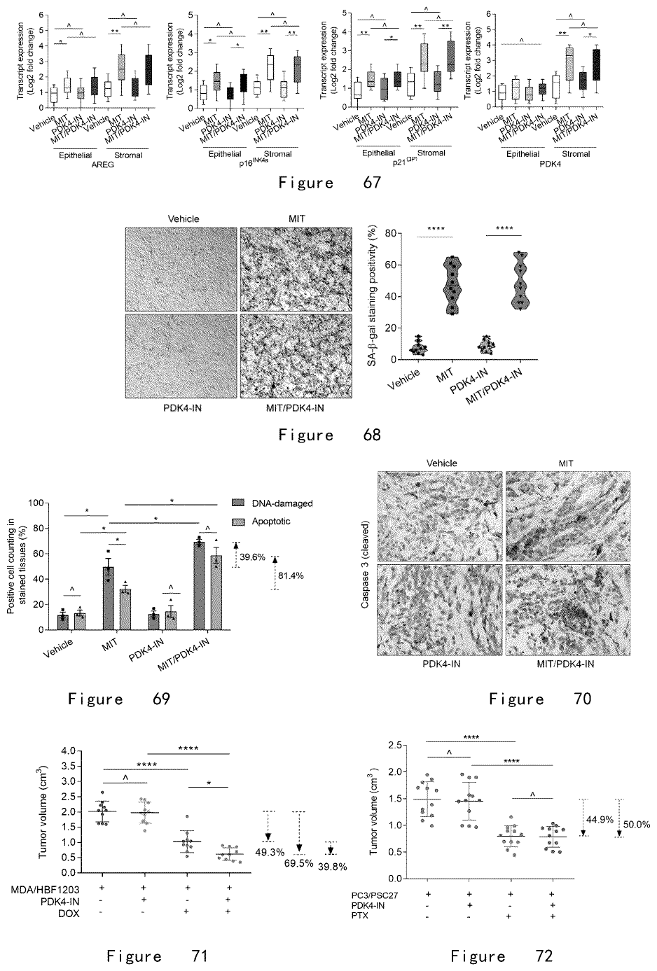

By analysis, the inventors observed that there was a considerable upregulation of typical SASP factors such as IL-6, CXCL8, MMP3, SPINK1 and AREG, accompanied by expression of typical senescence markers p16^{INK4a} and p21 in xenografting cells, implying development of in vivo cellular senescence and SASP expression in vivo (FIGS. 66-67, the lowest value was used as normalization baseline per factor). Although PDK4-IN alone neither induced nor affected cellular senescence, as evidenced by its results as a single agent or in combination with MIT (FIGS. 66-67). Although p16INK4a and p21CIP1 expression resulted in induced senescence of cancer cells in tumor-bearing sites of mice, the inventors did not observe a typical and full-spectrum SASP in these epithelial cells. This result is largely consistent with former findings of the inventors. Of note, PDK4 expression was significantly induced in stromal cell populations, but not in their epithelial counterparts (FIG. 67), basically in line with in vitro datasets of the inventors. Histological staining indicated elevated SA-β-gal positivity in tumor tissues of mice that experienced MIT or MIT/PDK4-IN treatment, proving that animals of these experimental groups had undergone extensive cellular senescence (FIG. 68). In contrast, PDK4-IN per se neither induced senescence nor inhibited senescence, consistent with the characteristics of this drug accordingly. This drug presumably neither targets DNA nor damages other macromolecules (FIGS. 66-68).

Considering the primary in vivo expressive results with MIT and/or PDK4-IN treatment, the inventors next asked how pharmacologically targeting PDK4 could enhance the therapeutic response of tumors. To disclose the possible mechanism(s), the inventors chose to dissect tumors from animals 7 days after initiation of treatment, a timepoint right before the development of resistant colonies. In contrast to the vehicle, MIT per se caused substantial DNA damage and apoptosis in cancer cells (FIG. 69). Although PDK4-IN alone neither caused typical DDR nor induced cell apoptosis, it showed prominent efficacy in enhancing these therapeutic indices upon combination with MIT. IHC staining disclosed increased caspase 3 cleavage, a canonical apoptosis indicator, upon MIT administration, with the tendency further enhanced by PDK4-IN (FIG. 70).

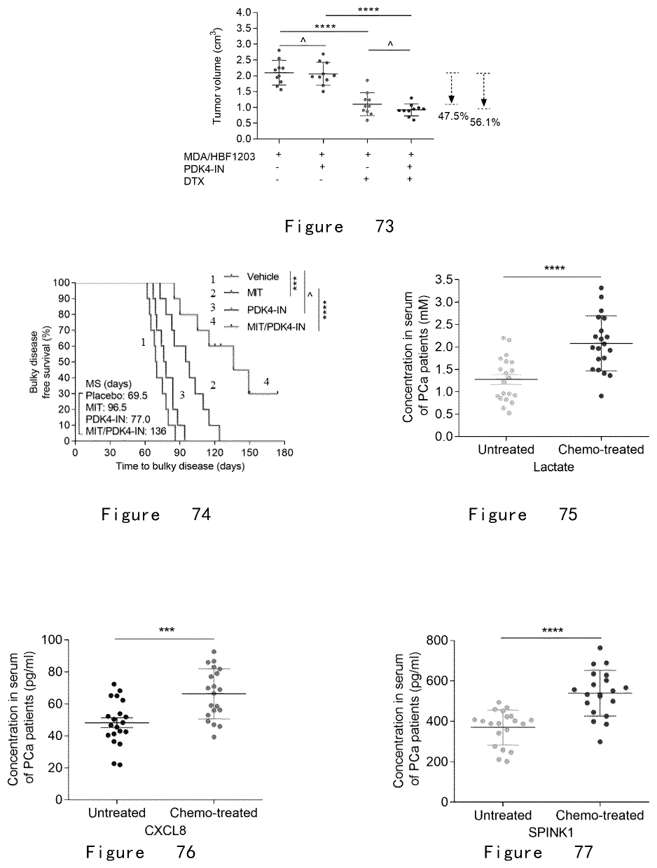

To assess the in vivo effects of chemotherapeutic drugs and/or PDK4-IN treatment in different tumor types, the inventors subsequently generated xenografts composed of breast cancer cell line MDA-MB-231 and breast stromal cell line HBF1203 to replicate the preclinical intervention observed in prostate cancer xenografts. The results showed that when PDK4-IN was combined with doxorubicin (DOX), a further decline of tumor volume was observed (39.8% reduction) on the basis of treating with doxorubicin (DOX); the tumor size was reduced by 69.5% in total compared to the control group (FIG. 71). However, when the inventors replaced the chemotherapeutic drugs with non-genotoxic drugs such as paclitaxel (PTX) or docetaxel (DTX), they were unable to replicate the effects observed in prostate cancer and breast cancer (FIGS. 72, 73). This indicates that significant and desirable effects can only be achieved through combined treatment with specific types of chemotherapeutic drugs and PDK4-IN.

Next, the inventors assessed tumor progression consequence by comparing the survival rate of different animal groups in a time-extended preclinical cohort. During tumor surveillance, bulky disease was considered once the tumor burden became prominent (size >2,000 mm³). Mice receiving MIT/PDK4-IN combinational treatment displayed the most prolonged median survival, gaining a 40.9% longer survival compared to those treated by MIT only (FIG. 74, green versus blue). However, PDK4-IN treatment alone did not achieve significant benefits, as it conferred only marginal survival advantage (FIG. 74; brown versus red). Thus, targeting PDK4 alone affects neither tumor growth nor animal survival, whereas MIT/PDK4-IN co-treatment has the competence to significantly improve both parameters.

### Example 7. Elevated Senescence-Associated Serum Lactate Content Predicts Survival of Patients with Cancer in the Post-Treatment Stage

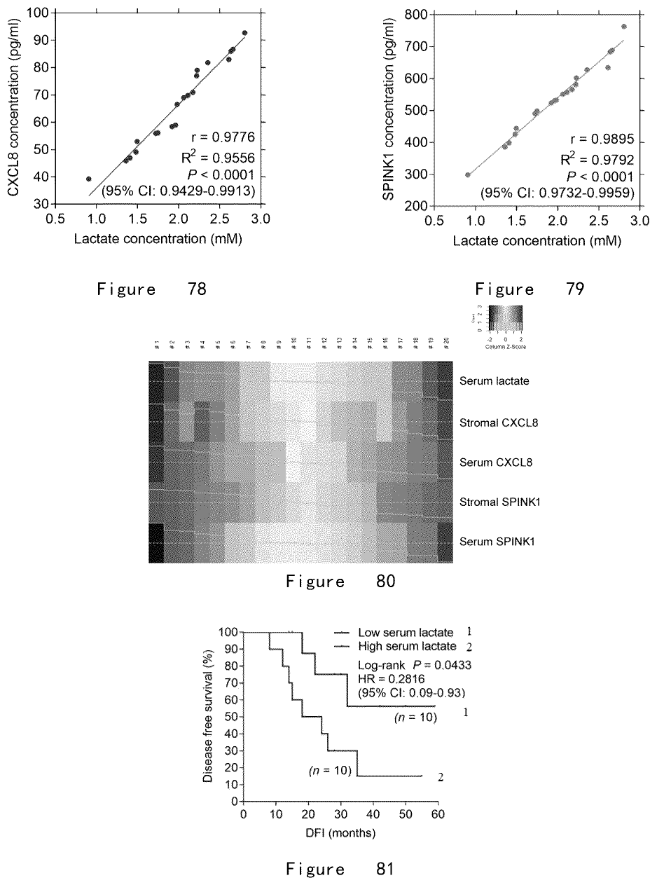

Despite the correlation of higher PDK4 expression in tumor loci with lower post-treatment survival (FIG. 14), whether the metabolite lactate is technically detectable in clinical and whether it can serve as a clinical marker, remains unclear to a large extent. To solve this question, the inventors acquired peripheral blood samples from patients with PCa, including one cohort that experienced standard neoadjuvant chemotherapy and the other that did not. ELISA assays of serum from chemo-treated patients revealed lactate levels in the treated cohort significantly higher than that of treatment-naive group (FIG. 75). The pattern was reproduced by a remarkable increase in serum level of CXCL8 and SPINK1, canonical SASP factors, in the same cohort of post-treatment patients (FIGS. 76-77). These data suggested that a circulating scale of lactate, a product of glucose metabolism, emerges in the peripheral blood by branched glycolytic metabolism and closely correlates with the in vivo levels of the SASP. This is associated with the abundance of senescent cells in situ following chemotherapeutic regimens, and the both are systemically traceable in the serum of treated patients with cancer. In addition, it would be interesting to determine whether there is a correlation between serum lactate levels and typical SASP factors such as CXCL8 and SPINK1 in the same individual's serum after clinical treatment. Subsequent analysis of ELISA data disclosed a significant and positive correlation between lactate and CXCL8, as well as between lactate and SPINK1 (FIG. 78-79). Thus, lactate production and SASP expression is mutually linked, largely resembling the clinical data derived from tumors per se from the inventors, especially the correlation between PDK4 induction and the SASP development as revealed (FIG. 13).

Subsequently, the inventors performed longitudinal analysis in both primary tumor foci and peripheral blood (20 chemo-treated patients randomly selected) and further continued extended researches. Surprisingly, cross-organ comparisons indicated a pronounced association between in-tissue expression and circulating level per factor, with lactate, CXCL8 and SPINK1 apparently varying in parallel either within the expression level or serum content in each individual (FIG. 80). Altogether, the date of the inventors showed that lactate represents a key TME-derived biological factor precisely mirroring development of an in vivo SASP and can be exploited to evaluate the SASP magnitude in post-treatment patients with cancer.

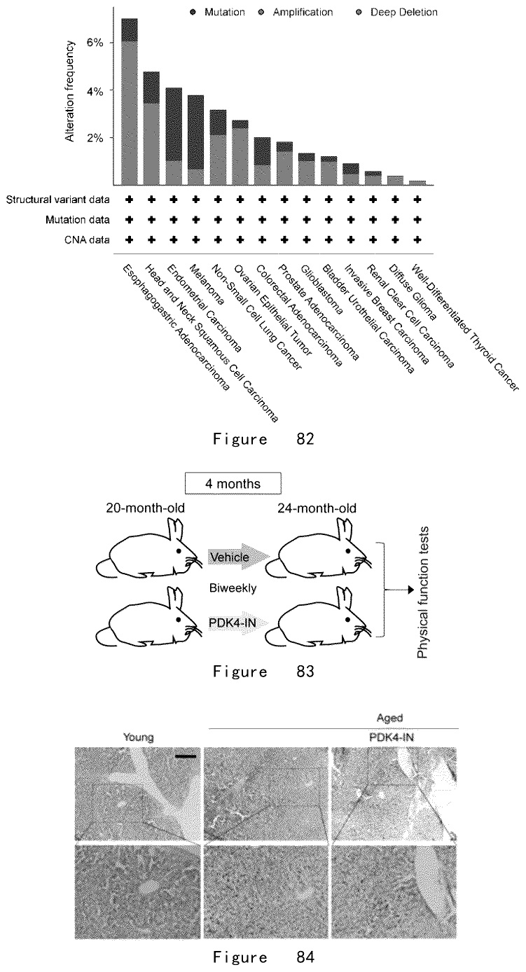

Clinical profiling subsequently uncovered a negative correlation between plasma level of lactate and post-treatment survival, further confirming the pathological impact of lactate. Lactic acid, as a molecule derived from the TME, can directly predict adverse outcomes once the tissue environment is subjected to irreversible damage induced in clinics (FIG. 81). As PDK4 is subject to frequent mutation, amplification and deep deletion as disclosed by TCGA pan-cancer atlas studies (querying 22,179 patients and 22,802 samples in 36 clinical studies) recording global genomic data from multiple cancer types (FIG. 82). The molecule represents an important predictor of treatment progression in clinical oncology. Therefore, routine monitoring of lactate, the major metabolic product associated with enhanced glycolysis-associated PDK4 overexpression, is of diagnostic significance. Especially in the condition of stromal cell senescence, non-invasive approaches such as liquid biopsy can offer a novel, practical, and accurate strategy for clinical management and prevention of advanced pathologies in clinical oncology.

### Example 8. Targeting PDK4 in the Late Stage Alleviates Physical Dysfunctions and Extends Lifespan in Mice

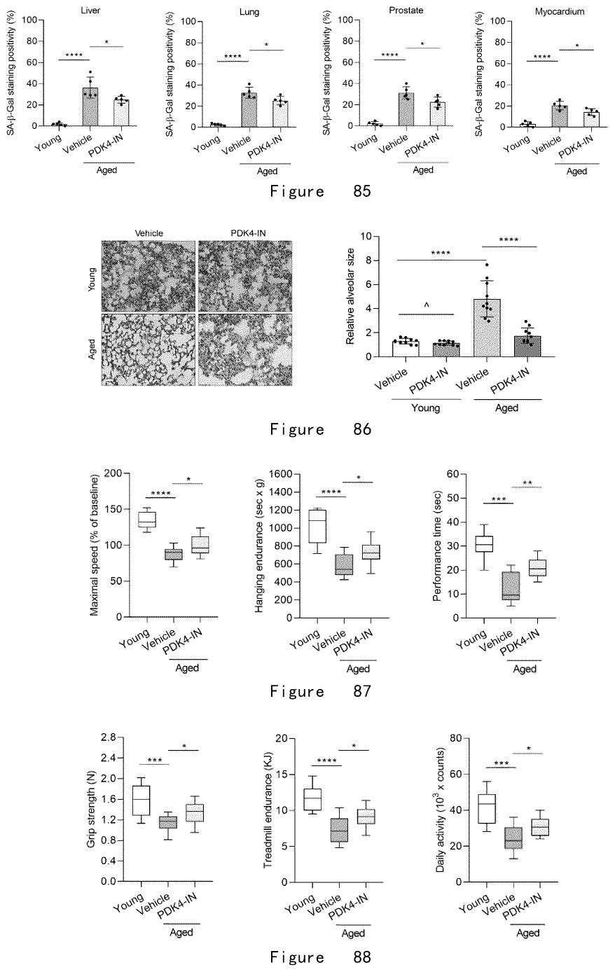

With the increasing global senescent trend in recent years, drugs that can effectively alleviate the senescent process have become a common interest among scientists and pharmaceutical companies worldwide. The inventors queried whether manipulation of PDK4 activities can control various senescence-associated phenotypes. Therefore, the inventors chose to treat normal 20-month-old wild-type mice with PDK4-IN (PDK4-IN 10 mg/kg via intraperitoneal injection once every 2 weeks) for 4 months, after which physical function or physical ability was experimentally determined in mice with natural senescence (FIG. 83). Histological evaluation disclosed emerging senescent cells in solid organs, as reflected by elevated SA-β-gal positivity in liver, lung, prostate and myocardial tissues of aged animals, changes that were partially reversed after treatment with PDK4-IN (FIGS. 84-85).

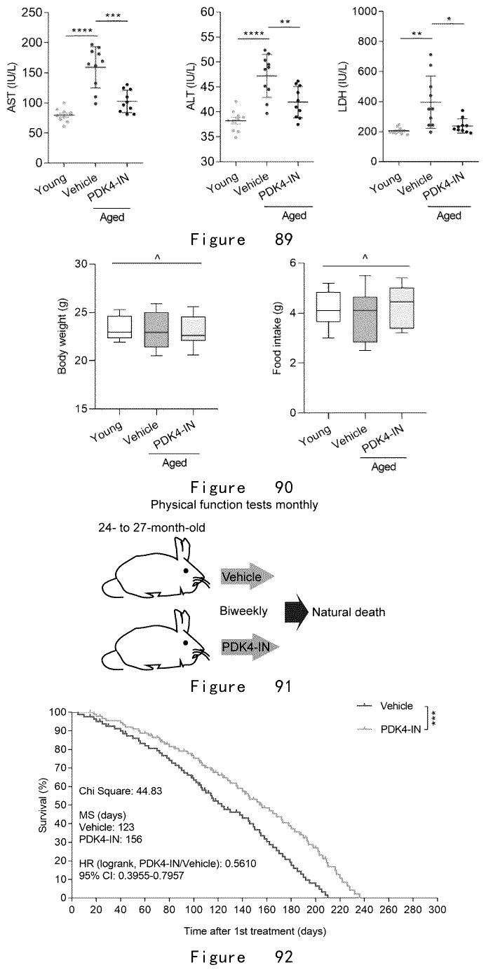

Notably, compared with young mice, senescence-associated health issues, such as increased alveolar volume, decreased grip strength and diminished motor skills in elderly (20-month-old) mice, were significantly improved (FIGS. 86-88). PDK4-IN exhibited significant protective and ameliorative effects on these physiological indicators and overall organismal functions. For instance, PDK-IN effectively controlled age-dependent expansion of alveoli, a pathological alteration associated with impaired lung function (FIG. 86). The data from the inventors suggest that the in vivo activity of PDK4 plays a crucial role in the structural abnormalities observed in lung tissues during senescence, with airway changes and alveolar abnormalities potentially leading to reduced respiratory function and the eventual development of chronic lung diseases during the senescent process. Furthermore, the inventors observed a significant reduction in serum lactate levels in aged animals receiving PDK4-IN treatment, indicating a key pathological contribution of PDK4 in regulating lactate production in these aged animals.

Compared to young animals, many aged mice exhibited an increasing trend in the occurrence of liver dysfunction, as evidenced by elevated levels of serum alanine aminotransferase (ALT), aspartate aminotransferase (AST), and lactate dehydrogenase (LDH) (FIG. 89). However, mice treated with PDK4-IN significantly avoided these changes, indicating that inhibiting PDK4 effectively improved the pathological mechanisms in the liver. Although PDK4-IN demonstrated efficacy in inhibiting liver dysfunction, the body weight and food intake levels of aged mice were essentially unaffected (FIG. 90), suggesting that the approach targeting PDK4 was safe in these animals.

To simulate the state of late life in humans, the inventors chose mice aged 24 to 27 months (equivalent to the human age range of 75-90 years). These mice were administered either Vehicle or PDK4-IN every two weeks, with continuous monitoring of their survival and recording of maximum lifespan (FIG. 91). After the preclinical stage of treatment in these mice, the data showed that both PDK4-IN and intraperitoneal administration significantly extended the lifespan of mice during the intervention period (P<0.001) (FIG. 92). These results suggest that targeting PDK4 and inhibiting the abnormal generation of lactate in vivo is an effective way to actively intervene in senescence and reduce the detrimental effects of senescent cells on organismal lifespan.

Above-described examples only show several embodiments of the present disclosure, which are described specifically and in detail. However, it should not be understood as a limiting patent scope of the present disclosure. It should be noted that those skilled in the art can make some adjustments and improvements without departing from the concept of the present disclosure and all these forms are within the scope of protection of the present disclosure. Therefore, the scope of patent protection in the present disclosure should be determined by the appended claims. Simultaneously, each reference provided herein is incorporated by reference to the same extent as if each reference was individually incorporated by reference.

1. A
method for inhibiting drug resistance of
tumors, suppressing tumors, or inhibiting or eliminating the SASP, comprising administration of a PDK4 down-regulator to required subjects, optionally the PDK4 down-regulator is administrated in combination with chemotherapeutic drugs.

2. (canceled)

3. A combination, a pharmaceutical combination or a drug kit for suppressing tumors, comprising: a down-regulator of PDK4 and a chemotherapeutic drug.

4. The method according to claim 1, wherein, the tumor is a tumor with drug resistance; said tumor with drug resistance after exposure to genotoxic drugs or exhibiting the SASP in the tumor microenvironment.

5. The method according to claim 4, wherein, the SASP is a SASP caused by DNA damage; the DNA damage is a DNA damage caused by a chemotherapeutic drug; the chemotherapeutic drug comprises a genotoxic drug;
and/or, the SASP is a hypermetabolic phenotype, the hypermetabolic phenotype is a phenotype with enhanced glycolysis, and/or increased TCA activities, and/or enhanced ATP-boosting oxidative phosphorylation; more preferably, the hypermetabolic phenotype is a phenotype with an increased citrate/pyruvate ratio, elevated levels of metabolites, increased expression or activity of enzymes in metabolic processes, perturbations in mitochondrial ultrastructure, significantly increased extracellular acetone and lactate production and extracellular acidification rate, increased oxygen consumption rate, and elevated ATP production, basal respiration and maximal respiration.

6. The method according to claim 1, wherein, the down-regulator of PDK4 comprises a substance down-regulating PDK4 activities or a substance down-regulating expression levels of PDK4, increasing the stability of PDK4 or reducing the effective time of PDK4.

7. The method according to claim 1, wherein, the chemotherapeutic drug is a chemotherapeutic drug that induces drug resistance of tumor after administration; comprising a genotoxic drug.

8. The pharmaceutical combination or drug kit according to claim 3, wherein, the weight ratio of PDK4 down-regulator to chemotherapeutic drug is 1:1˜1:200, preferably 1:2˜1:100, more preferably 1:5˜1:50.

9. The pharmaceutical combination or drug kit according to claim 3, wherein, the final concentration of PDK4 down-regulator is 0.1˜100 M, preferably 0.5˜50 M, more preferably 1˜10 M; or the final concentration of the chemotherapeutic drug is 0.01˜100 M, preferably 0.05˜80 M, more preferably 0.1˜50 M.

10. A method for preparing a pharmaceutical combination or drug kit for inhibiting tumors, comprising: mixing the PDK4 down-regulator and the chemotherapeutic drug; or placing the PDK4 down-regulator and the chemotherapeutic drug in the same drug kit.

11. A method for screening a potential substance for enhancing the anti-tumor effect of chemotherapeutic drugs, wherein the method comprises:
(1) providing a system of tumor microenvironment, wherein the system comprises tumor cells and stromal cells;
(2) treating the system of (1) with a chemotherapeutic drug, inducing a SASP in the tumor microenvironment;
(3) adding the candidate substance to the system in (2) and observing its effect on the tumor microenvironment system;
if it shows specifically-targeted clearance of senescent cells in a tumor microenvironment and/or promotes the growth of stromal cells, then it is a potential substance for enhancing the anti-tumor effect of chemotherapeutic drugs.

12. A method for screening a potential substance for inhibiting drug resistance of tumor or inhibiting the SASP, wherein the method comprises:
(1) treating an expression system by a candidate substance, wherein the system expressing PDK4; and
(2) detecting the system to observe the expression or activity of PDK4; if the expression or activity of PDK4 is down-regulated statistically by the candidate substance, then the candidate substance is the potential substance for inhibiting drug resistance of tumor or inhibiting the SASP.

13. A method for diagnosis or prognosis of drug resistance of tumor, comprising using a reagent specifically recognizing or PDK4, when the expression of PDK4 is higher than the specified threshold, it can be considered for treatment with a PDK4-inhibiting regimen.

14. The method according to claim 13, wherein, the diagnostic reagents comprise: a binding molecule specifically binding to PDK4 protein, a primer specifically amplifying the PDK4 gene, a probe specifically recognizing the PDK4 gene, a chip specifically recognizing the PDK4 gene, a binding molecule specifically binding to lactate, a probe specifically recognizing lactate, or an enzyme specifically monitoring lactate levels.

15. (canceled)

16. The method according to claim 4, wherein, the tumor is selected from: prostate cancer, breast cancer, lung cancer, colorectal cancer, gastric cancer, liver cancer, pancreatic cancer, bladder cancer, skin cancer, or kidney cancer.

17. The method according to claim 6, wherein, the down-regulator comprises: a chemical molecular antagonist or inhibitor targeting PDK4, a reagent knocking out or silencing PDK4, or a proteasome.

18. The method according to claim 6, wherein, the down-regulator of PDK4 is PDK4-IN; or the reagent knocking out or silencing PDK4 comprises: an interfering molecule that specifically interferes with the expression of the encoding sequence of PDK4, a CRISPR gene-editing agent targeting PDK4, a homologous recombinant agent or an agent for site-directed mutagenesis, wherein the agent for site-directed mutagenesis brings a loss-of-function mutation to the PDK4.

19. The method according to claim 7, wherein, the chemotherapeutic drug is: mitoxantrone, doxorubicin, bleomycin, vinblastine, paclitaxel, docetaxel, satraplatin, cisplatin, carboplatin, daunorubicin, nogamycin, aclarubicin, epirubicin, doxorubicin, cytarabine, capecitabine, gemcitabine, or 5-fluorouracil.

20. The method according to claim 11, wherein, in step (2), it also comprises:
administering a PDK4 down-regulator before, during or after inducing the SASP in the tumor microenvironment;
and/or, in step (3), it also comprises: if the candidate substance statistically promotes a down-regulator of PDK4 or an encoding gene thereof to clear senescent cells in the tumor microenvironment and/or promotes the growth of stromal cells, then the candidate substance is a potential substance that can be used in combination with the down-regulator of PDK4 or the encoding gene thereof to inhibit tumors.

21. The method according to claim 12, wherein, the step (1) comprises: in the testing group, adding the candidate substance into the expression system; and/or
the step (2) comprises: detecting the system to observe the expression or activity of PDK4 and compared to the control group, wherein the control group is an expression system without adding the candidate substances; if the expression or activity of PDK4 is down-regulated statistically by the candidate substance, then the candidate substance is the potential substance for inhibiting drug resistance of tumor or inhibiting the SASP.
